(19) United States
(12) Patent Application Publication
(10) Pub. No.: US 2007/0054899 A1
(43) Pub. Date: Mar. 8, 2007

(54) Aromatase inhibitor compounds and uses thereof

(76) Inventors: Chang-Ha Park, Ansan City Kyunggi-Do (KR); Said Yous, Loos (FR); Celine Nativelle-Serpentini, Thorigny Sur Vire (FR); Gilles-Eric Seralini, Janville (FR); Soon-Jae Chang, Suwon City Kyunggi-Do (KR); Daniel Lesieur, Gondecourt (FR)

Correspondence Address: FULBRIGHT & JAWORSKI L.L.P., 600 CONGRESS AVE., SUITE 2400, AUSTIN, TX, 78701, US

(21) Appl. No.: 10/595,242

(22) PCT Filed: Sep. 24, 2004
(86) PCT No.: PCT/FR04/50471

§ 371 Date: Aug. 23, 2006

(30) Foreign Application Priority Data
Sep. 29, 2003 (FR) 0311397

(51) Int. Cl. A61K 31/5415; A61K 31/538; C07D 417/02; C07D 413/02; C07D 419/02

(52) U.S. Cl. 514/224.2; 514/230.5; 544/1; 544/105; 544/52; 514/183

(57) ABSTRACT

The invention concerns the use of a compound of formula (I) inhibitor of aromatase for the preparation of a pharmaceutical formulation intended for treatment of cancer or psoriasis. It equally relates to compounds of formula (I), notably for their use as active ingredients of a medication.

### FIELD OF THE INVENTION

The present invention relates to new compounds inhibitory to aromatase and their utilization in the medical field, and more specifically in the prevention and treatment of cancer, particularly breast cancer, or of psoriasis.

### PRIOR ART

Certain derivatives of the benzazolinones and particularly of benzoxazolinone, have already been described for their gonadotrope, antiproliferative and immunomodulating properties (BERGER et al. 1981 BUTTERSTEIN, et al. 1988; SCHADLER et al. 1988).

In the course of the last ten years, the azole class of compounds (imidazoles and triazoles) has shown activity inhibitory to aromatase that has led to their use in the treatment of certain breast cancers (KUIJPERS et al. 1998; SCHADLER et al. 2001; BRODIE et al. 2002).

It has been shown that, in mammals, and in particular in humans, oestrogens are synthesised from androgens by enzyme catalysis with aromatase. It is readily acknowledged that inhibition of aromatase is useful in the prevention or the treatment of disorders and pathologies associated with oestrogens in mammals, such as breast cancer. The other diseases associated with oestrogens which can be treated with a compound inhibitory to aromatase include endometriosis, cancer of the neck of the uterus, cancer of the ovaries, polycystic ovarian syndrome. It is also considered that an aromatase inhibitor compound is useful for birth control. More particularly, in the case of breast cancer, it is said that an aromatase inhibitor compound can be advantageously used, as a replacement for typical surgical treatment such as ovariectomy or adrenalectomy.

It is also known that an aromatase inhibitor compound is useful in the prevention or treatment of cancer of the prostate.

The benefit has also been shown of using an aromatase inhibitor compound for the treatment of psoriasis.

Notably olefinic compounds inhibitory to aromatase comprising one or several heterocycles are described in the European patent application n^{o} EP-299 683. Other aromatase inhibitor compounds, such as the compound designated “TAN-931”, have been described in the European patent application n^{o} EP-342 665. Also the aromatase inhibitor compounds diarylalkyl heterocyclics such as those described in the PCT application n^{o} WO 94/13645 or in the PCT application n^{o} WO 02/087571 are known. Equally the heterocyclic derivatives of aralkyl aromatase inhibitors, as described in the European patent application n^{o} EP-296 749 are known. Also described are the aromatase inhibitor compounds composed of imidazolyl or triazolyl derivatives of pyrimidine or of dihydropyridine substituted by a phenyl, as in the applications for European patent n^{o} EP-755 931 and n^{o} EP-533 504, or again as in the PCT application n^{o} WO 90/06923. Condensed tricyclic aromatase inhibitors have also been described in the European patent application n^{o} EP-360 324.

Nevertheless, there exists a need, in the state of the art, for new compounds inhibitory to aromatase, useful in therapy, which offer good properties for inhibition of this enzyme, and which are without toxicity, both in vitro and in vivo.

### DETAILED DESCRIPTION OF THE INVENTION

The present invention concerns the preparation of new azole derivatives of various benzazolinones, (benzoxazolinone, benzothiazolinone, benzoselenazolinone, benzoxazinone, benzothiazinone and indolinone), which possess aromatase inhibitory properties and remarkable anti-cancer and anti-psoriasis properties.

The object of the invention is the utilization of a compound of the formula (I) below:

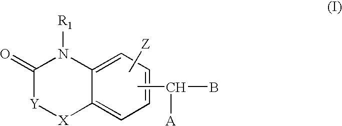

wherein:

R₁ represents an atom of hydrogen or a linear or branched alkyl (C₁-C₆), alkenyl (alkene) (C₁-C₆), or alkynyl (alkyne) (C₁-C₆) radical,

X represents an atom of oxygen, sulphur or selenium;

Y represents a single bond or a CH₂ group, possibly substituted by one or two lower alkyl groups.

Z represents an atom of hydrogen or halogen, or a hydroxy or a linear or branched alkoxy group.

A represents an imidazole, triazole or tetrazole nucleus,

B represents a group selected from the groups phenyl, naphthyl, biphenyl or also a monocyclic or bicyclic heteroaryl group having 5 to 10 bonds and containing 1 to 3 heteroatoms.

the groups phenyl, naphthyl, biphenyl and heteroaryl being non-substituted or substituted with 1 to 3 groups selected from alkyl (C₁-C₆), alkoxy (C₁-C₆), carboxy, formyl, amino, amido, ester, nitro, cyano, trifluoromethyl, or atoms of halogen,

as well as enantiomers and diastereomers of compounds of formula (I), as well as the salts from the addition to an acid or to a pharmaceutically acceptable base of compounds of the formula (I),

for preparation of a pharmaceutical formulation intended for treatment of cancer or psoriasis.

The term “heteroaryl” means, according to the invention, all mono- or bi-cyclic groups comprising 5 to 10 bonds and 1 to 3 heteroatoms selected from oxygen, nitrogen and sulphur. In the meaning of the invention, heteroaryl groups containing 5, 6, 7, 8, 9, or 10 bonds are included. The heteroaryl groups comprising 1, 2 or 3 heteroatoms selected from among oxygen, nitrogen and sulphur are included.

The groups aryl and heteroaryl B of a compound of formula (I) such as defined herein can be substituted with 1, 2 or 3 groups selected from among alkyl (C₁-C₆), alkoxy (C₁-C₆), carboxy, formyl, amino, amido, ester, nitro, cyano, trifluoromethyl, or atoms of halogen. Therefore, in the meaning of the invention, the groups C₁-, C₂-, C₃-, C₄-, C₅-, and C₆-alkyl, as well as the groups C₁-, C₂-, C₃-, C₄-, C₅-, and C₆-alkoxy are included.

All salts from the addition of a compound of formula (I) to a pharmaceutically acceptable acid are included in the invention. Among the acids pharmaceutically acceptable, are cited preferably, but not imitatively, the hydrochloric, hydrobromic, sulphuric, acetic, trifluoroacetic, lactic, succinic, fumaric, citric, oxalic or methane sulphonic acids.

All salts from the addition of a compound of formula (I) to a pharmaceutically acceptable base are included in the invention. Among the pharmaceutically acceptable bases, are cited for preference, but not imitatively, sodium hydroxide, potassium hydroxide or triethylamine.

It has been shown according to the invention that compounds of the formula (I) defined herein are highly innocuous, in vitro as well as in vivo. Thus, it is shown that compounds of the formula (I) are not cytotoxic in vitro. It has also been shown that a compound of formula (I) presents no danger, even at a high dose, when it is administered to an individual.

It has also been shown according to the invention that compounds of formula (I) are good aromatase inhibitors. Certain compounds of the formula (I) present an inhibitory power IC₅₀ of the order of 1 nM.

Equally it has been shown that compounds of the formula (I) are active in vivo, as illustrated by their capacity to inhibit and, in some cases, block, the uterine hypertrophy induced by androstenedione.

In general, the preferred compounds of formula (I) according to the invention are the compounds n^{o} 1 to 51, described in examples 1 to 51, the structure of which is described in detail in Table IV.

The first family of preferred compounds of formula (I) according to the invention is comprised of compounds for which the group B is selected from among:
unsubstituted benzene or benzene substituted in the meta or para position by a group selected from among the groups cyano or nitro, or by a chlorine atom;
a heterocyclic pyridine.

A second family of preferred compounds of formula (I) according to the invention is comprised of compounds for which the group R1 represents a hydrogen atom or a methyl group.

A third family of preferred compounds of formula (I) according to the invention is comprised of compounds for which the group Z represents a hydrogen atom or a methoxy group.

A fourth family of preferred compounds of formula (I) according to the invention is comprised of compounds for which the group A represents a 1,3-imidazolyl or 1,2,4-triazolyl group.

A fifth family of preferred compounds of formula (I) according to the invention is comprised of compounds for which, simultaneously:

(i) group B is selected from among:
unsubstituted benzene or benzene substituted in the meta or para position by a group selected from among the groups cyano or nitro, or by a chlorine atom;
   a heterocyclic pyridine;

(ii) group R1 represents a hydrogen atom or methyl group;

(iii) group Z represents a hydrogen atom or methoxy group; and

(iv) group A represents a 1,3-imidazolyl or 1,2,4-triazolyl group.

Another object of the invention is an aromatase inhibitor compound, such as defined herein, for use as an active ingredient of a medicine.

The invention equally concerns, as a new compound, any of the compounds of formula (I) as described in the present description.

In their role for therapy, the compounds of formula (I) are particularly useful when they are used in manufacturing a pharmaceutical formulation intended for the prevention or the treatment of disorders and pathologies associated with oestrogens in mammals, such as breast cancer, endometriosis, cancer of the neck of the uterus, ovarian cancer, prostate cancer or polycystic ovarian syndrome.

A compound of formula (I) is equally advantageously used to manufacture a pharmaceutical formulation intended for treatment of psoriasis.

The present invention also has for an object a pharmaceutical formulation characterized in that it comprises at least one compound of general formula (I) described herein, in association with at least one excipient selected from among the group consisting of pharmaceutically acceptable excipients.

To make a pharmaceutical formulation according to the invention, those skilled in the art could advantageously refer to the last edition of the European Pharmacopoeia or the United States Pharmacopoeia (USP).

Those skilled in the art could particularly advantageously refer to the 4^{th} edition “2002” of the European Pharmacopoeia, or edition USP 25-NF20 of the American Pharmacopoeia (U.S. Pharmacopoeia).

Advantageously, a pharmaceutical formulation such as defined is adapted to daily administration, preferably by the oral route or topically, of a quantity of the compound of formula (I) ranging from 1 μg to 10 mg and preferably from 0.5 mg to 10 mg.

Advantageously, a pharmaceutical formulation such as defined herein is adapted for daily systemic administration of a quantity of the compound of formula (I) ranging from 0.5 mg to 10 mg.

When the formulation according to the invention comprises at least one pharmaceutically acceptable excipient, this is in particular an excipient appropriate for administration of the formulation by the topical route and/or an excipient appropriate for administration of the formulation by the oral route.

Administration by the systemic route is preferred for a pharmaceutical formulation comprising a compound of formula (I), for example by the oral route, for the prevention or treatment of a cancer.

Administration by the topical route is preferred for a pharmaceutical formulation comprising a compound of formula (I) for the treatment of psoriasis.

The invention also relates to a method for treating cancer in a patient, preferentially a cancer associated with oestrogens, said method comprising a step in the course of which the patient is administered a therapeutically effective quantity of a compound of formula (I) or a pharmaceutical formulation containing a compound of formula (I).

The invention also relates to a method for preventing cancer in a patient, preferentially a cancer associated with oestrogens, said method comprising a step in the course of which the patient is administered a therapeutically effective quantity of a compound of formula (I) or a pharmaceutical formulation containing a compound of formula (I).

The invention also relates to a method for treating psoriasis in a patient said method comprising a step in the course of which the patient is administered a therapeutically effective quantity of a compound of formula (I) or a pharmaceutical formulation containing a compound of formula (I).

The present invention equally relates to the process for obtaining compounds of formula (I) characterized in that the starting product used is a compound of formula (II).

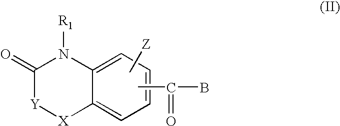

wherein R₁, X, Y, Z and B have the same meaning as in formula (I) obtained according to one of the protocols described by BONTE et al. 1974; AICHAOUI et al. (1990, 1991 and 1992), MOUSSAVI et al. (1989), SASTRY et al. (1988), and YOUS et al. (1994)

which is reduced to obtain a compound of formula (III). 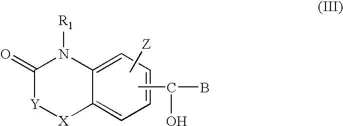
wherein R₁, X, Y, Z and B have the same meaning as in formula (I)

which is subsequently:

either treated with carbonyldiimidazole in order to obtain a compound of formula (I).

or treated with thionyl chloride to lead intermediately to a non-isolated compound of formula (IV). 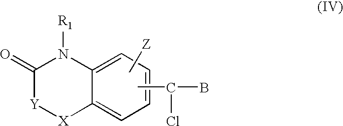
which is reacted with an azole derivative: imidazole, triazole or tetrazole, in order to obtain compounds of formula (I)

The preparatory separations of enantiomers of certain compounds selected from among the most active were achieved with the help of stationary phase chiral polysaccharide columns (cellulose or amylose) using a non-polar mobile phase.

The optical purity of each enantiomer isolated was then measured with the help of analytic columns of the same chiral stationary phase as those having performed the preparative separation and in the same operating conditions.

The materials first used in the previously described procedure are either commercial, or easily available to those skilled in the art following the literature and the preparation examples given hereunder.

For example, it is possible to prepare the compounds of is formula (IIIa) or (IIIb) 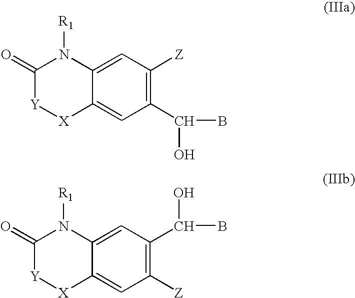
wherein R₁, X, Y, Z and B have the same meaning as in formula (I) by reaction of a compound of formula (V) 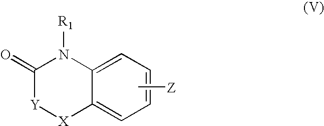
wherein R₁, X, Y, Z and B have the same meaning as in formula (I)

either with a chloride or an acid anhydride of formula B—COCl or (B—CO)₂O, in the presence of aluminium trichloride and of dimethylformamide

or with an acid of formula B—COOH, in the presence of polyphosphoric acid
to obtain a compound of formula (IIa) or (IIb) 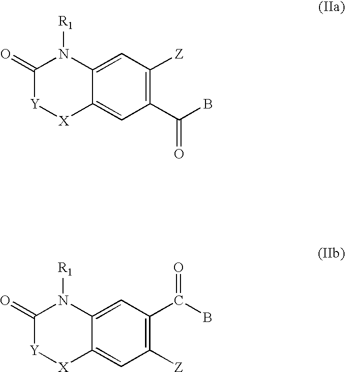
wherein R₁, X, Y, Z and B have the same meaning as in formula (I) which is reduced by sodium borohydride to obtain a compound of formula (IIIa) or (IIIb)

A further example of preparation of compounds of formula (I) consists in using the 4-acyl 2-aminophenols of formula (VI) 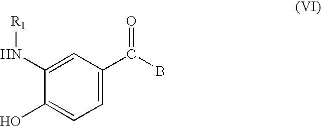
wherein R1, X, Y, Z and B have the same meaning as in formula (I) to obtain by heterocyclisation according to a protocol described by AICHAOUI et al. (1990) 5-acyl benzoxazolinones of formula (IIc) 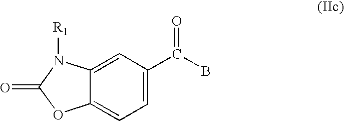
which are then subjected to the same reaction sequence as before.

Other routes of synthesis of compounds of formula (I) according to the invention are described in the examples and illustrated in FIGS. 4 and 5.

The present invention is further illustrated by the following figures and examples.

### DESCRIPTION OF THE DRAWINGS

FIG. 1 illustrates a first synthesis diagram of a compound of formula (I) according to the invention.

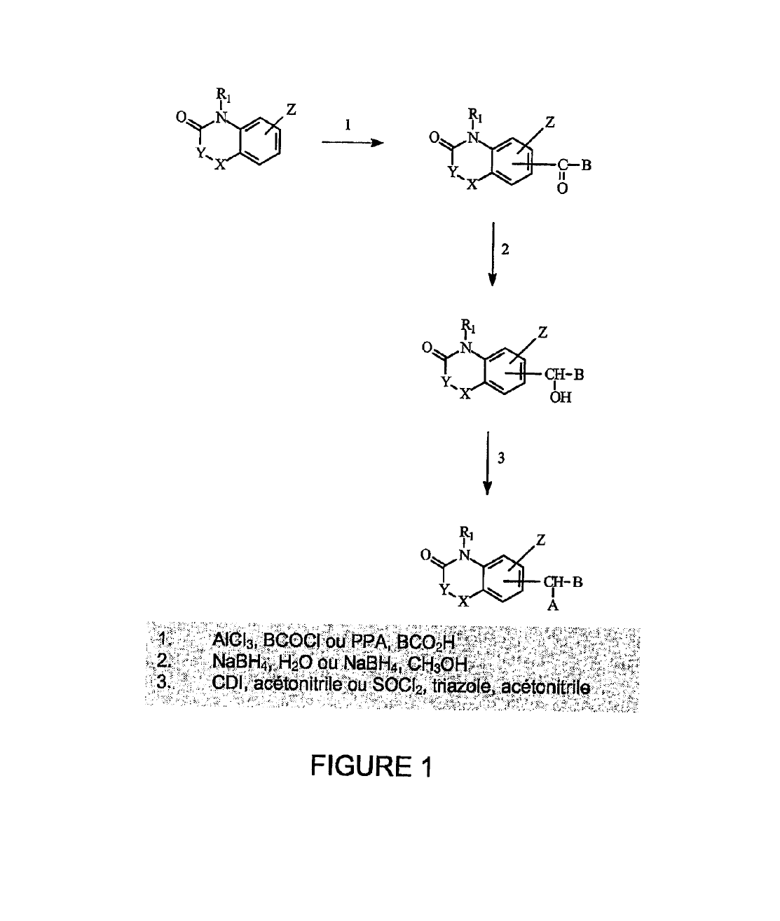

FIG. 2 illustrates a second synthesis diagram of a compound of formula (I) according to the invention.

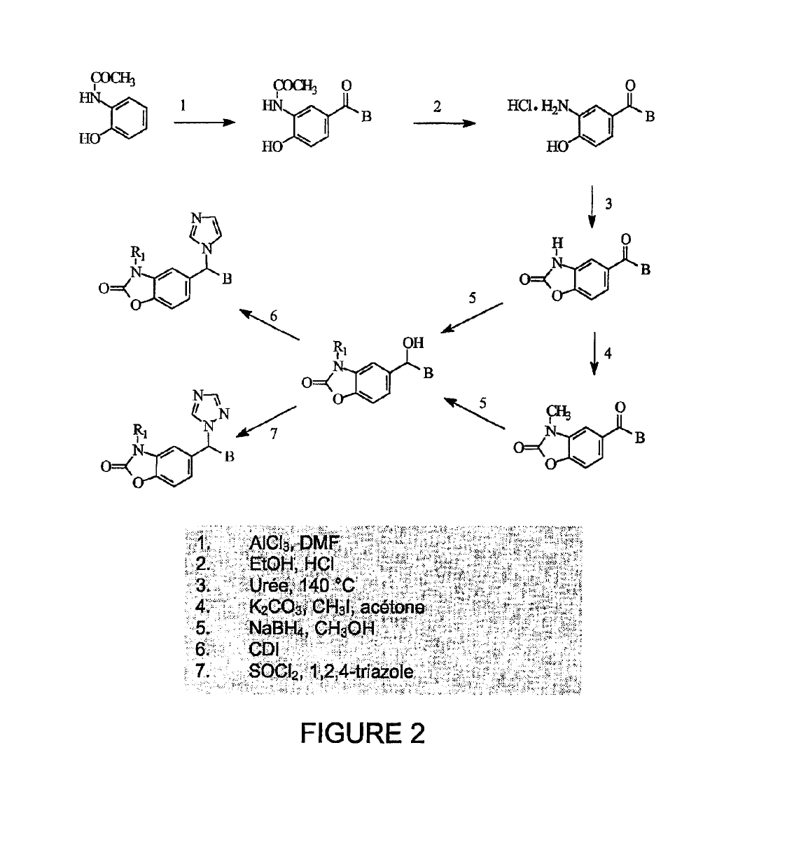

FIG. 3 illustrates a third synthesis diagram of a compound of formula (I) according to the invention.

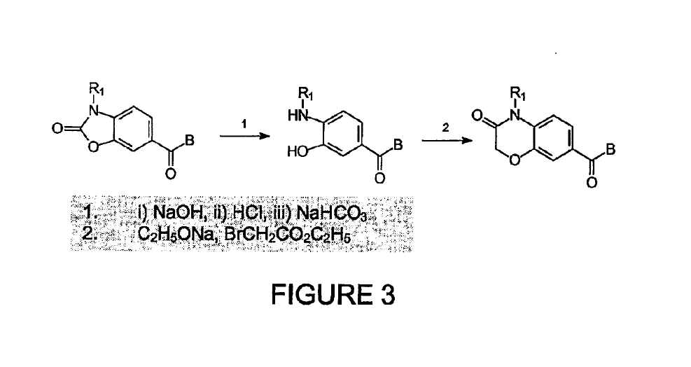

FIG. 4 illustrates a synthesis diagram of a compound of formula (I) according to the invention, of the 5-benzothiazolinone type.

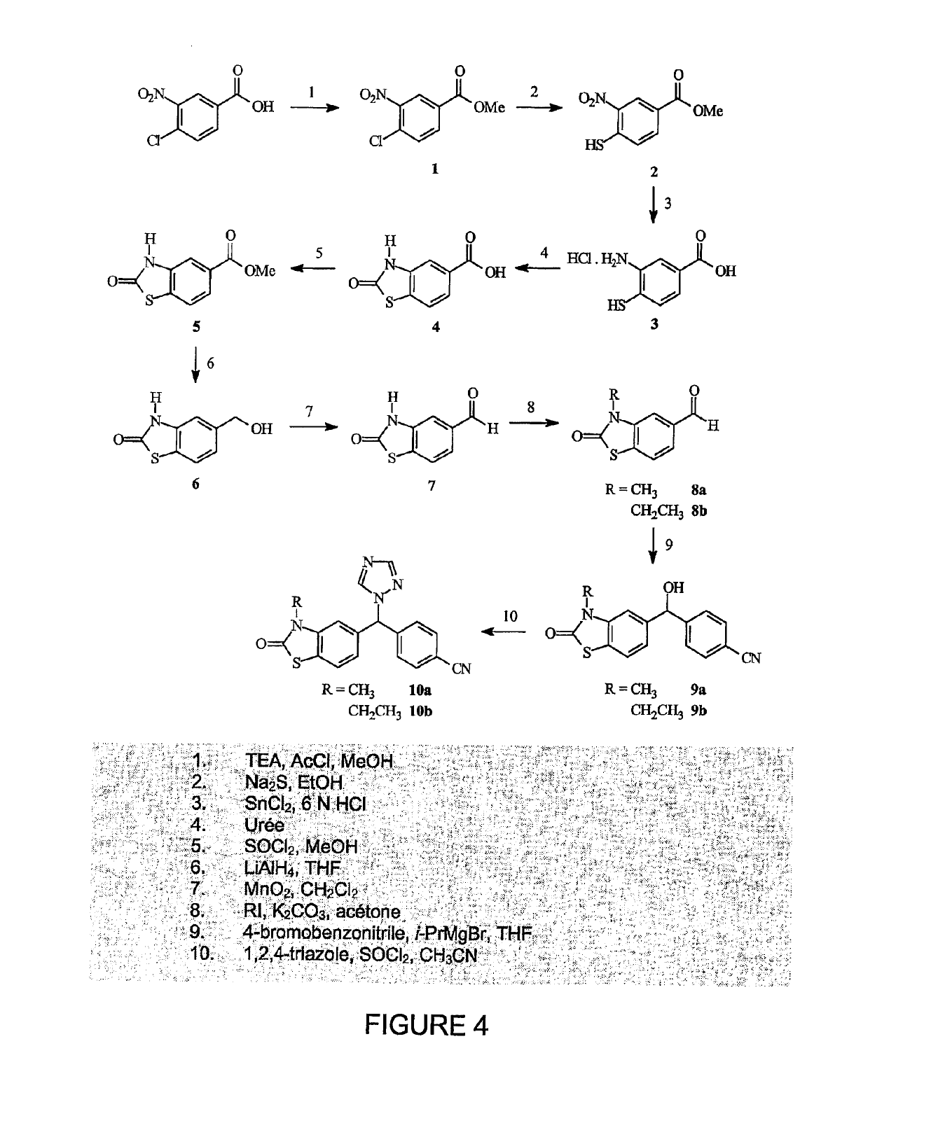

FIG. 5 illustrates a synthesis diagram of a compound of formula (I) according to the invention, of the 6-benzoselenazolinone type.

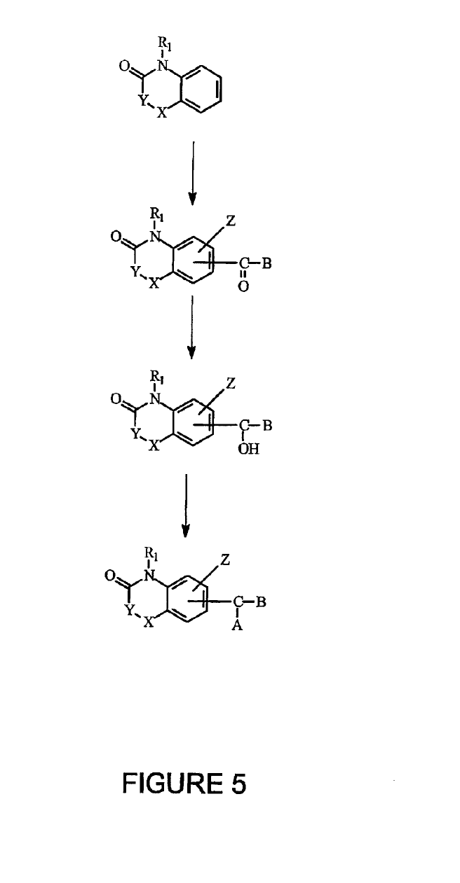

### EXAMPLES

The following embodiments illustrate the invention and do not limit it in any way. The following preparations lead to synthesis intermediates used in the invention preparation.

The products described in the “preparations” are not part of the invention. However their description facilitates the making of the compounds of formula (I) of the invention.

### A. General Method of Synthesis of Compounds of Formula (I) of the Invention

### A.1. Preparation 1: 6-Acyl benzazinones and 7-acyl-benzothiazinone (Table I-A)

The 6-acyl benzoxazolinones, benzothiazolinones, benzoxazinones, indolinones and 7-acyl-benzothiazinones and benzoselenazolinones are obtained from the corresponding benzazolinones according to two known procedures and using either chloride or acid anhydride in the presence of aluminium trichloride in methylformamide (Method B), or the organic acid itself in the presence of polyphosphoric acid (Method A) (AICHAOUI et al, 1992; BONTE et al, 1974; SASTRY et al, 1988; YOUS et al, 1994).

### A.2. Preparation 2: 5-Acyl benzoxazolinones (Table II).

The 5-acyl benzoxazolinones are prepared from 4-acyl-2-aminophenols according to the procedure described by AICHOUI et al, (1990).

### A.3. Preparation 3: 7-Acyl benzoxazinones (Table II)

The 7-acyl benzoxazinones are prepared from 5-acyl-2-aminophenols according to the procedure described by MOUSSAVI et al. (1989).

### A.4. Preparation 4: Hydroxyarylmethyl benzazinones (Table III-A)

Dissolve acyl benzazinone in methanol (R₁=alkyl, method A) or in an aqueous solution of sodium hydroxide (R₁=H, method B). Slowly add with stirring 2 equivalents of sodium borohydride then stir at ambient temperature for three hours and acidify with 6M hydrochloric acid. Spin out the precipitate, wash with water, dry and recrystallise in an appropriate solvent.

### B. Examples of Synthesis of Compounds of Formula (I)

### Example 1

### 6-[(4-Cyanophenyl)(1H-imidazol-1-yl)methyl]-1,3-benzoxazol-2(3H)-one

In 30 ml of acetonitrile, 5 mole of 6-[1-hydroxy-1-(4-cyanophenyl)methyl]-1,3-benzoxazol-2(3H)-one and 5 mmole of N,N′-carbonyldiimidazole are refluxed for 24 hours. The solvent is then evaporated under vacuum. The residue is triturated with 100 ml of water then acidified with 6M hydrochloric acid and extracted with diethyl ether. The aqueous phase is alkalised with a saturated solution of sodium carbonate then extracted twice with 100 ml of ethyl acetate. The organic phase is washed with water, dried on magnesium sulphate and vaporised. The residue obtained is purified by column chromatography. The fractions containing the product are vaporised and the residue obtained is triturated with petroleum ether before drying. F.° C.: 122-126° c.

### Examples 2 to 19

By proceeding as in example 1, but replacing the 6-[1-hydroxy-1-(4-cyanophenyl)methyl]-1,3-benzoxazol-2(3H)-one by the appropriate hydroxyarylmethyl benzazinone, the products in examples 2 to 19 (table IV) are obtained.

### Example 20

### 6-[(4-Cyanophenyl)(1H-1,2,4-triazol-1-yl)methyl]-3-methyl-1,3-benzothiazol-2(3H)-one

Thionyl chloride (15 mmol) is added to a solution of 1H-1,2,4-triazole (60 mmol) in acetonitrile (30 ml). The reaction mixture is stirred for 1 h at ambient temperature before being filtered. The solution obtained is added drop by drop to a solution of 6-[1-hydroxy-1-(4-cyanophenyl)methyl]-1,3-benzoxazol-2(3H)-one (4 mmol) in acetonitrile (10 ml). After 5 h of stirring at ambient temperature the solvent is evaporated under vacuum. The residue obtained is triturated with 100 ml of water then acidified with 6M hydrochloric acid and extracted with diethyl ether. The aqueous phase is alkalised with a saturated solution of sodium carbonate then extracted twice with 100 ml of ethyl acetate. The organic phase is washed with water, dried on magnesium sulphate and vaporised. The residue obtained is purified by column chromatography. The fractions containing the product are vaporised and the residue obtained is triturated with petroleum ether before drying. F.° C. 127-130° C.

### Examples 21 to 24

By proceeding as in example 20, but replacing the 6-[1-hydroxy-1-(4-cyanophenyl)methyl]-1,3-benzothiazol-2(3H)-one by an appropriate hydroxyarylmethyl benzazinone, the products in examples 21 to 24 (table IV) are obtained.

### Examples 25 to 43

Proceeding as in the preceding examples, one obtains similarly:

6-[1H-Imidazol-1-yl(phenyl)methyl]-1,3-benzoxazol-2(3H)-one (25). F ° C. 193-195° C.

6-[1H-Imidazol-1-yl(phenyl)methyl]-3-methyl-1,3-benzoxazol-2(3H)-one (26). F ° C.73-74° C.

6-[(4-Chlorophenyl)(1H-imidazol-1-yl)methyl]-3-methyl-1,3-benzoxazol-2(3H)-one (27). F.° C. 76-78° C.

3-Methyl-6-[phenyl(4H-1,3,4-triazol-4-yl)methyl]-1,3-benzoxazol-2(3H)-one (28). F.° C. 225-226° C.

3-Methyl-6-[phenyl(1H-1,2,4-triazol-1-yl)methyl]-1,3-benzoxazol-2(3H)-one (29). F.° C. 76-78° C.

5-[1H-Imidazol-1-yl(phenyl)methyl]-1,3-benzoxazol-2(3H)-one (30). F ° C 108-111° C

3-Methyl-5-[1H-imidazol-1-yl-(phenyl)methyl]-1,3-benzoxazol-2(3H)-one (31). F ° C. 133-135° C.

3-Methyl-5-[1H-1,2,4-triazol-1-yl(phenyl)methyl]-1,3-benzoxazol-2(3H)-one (32). F ° C. 135-138° C.

5-[(4-Chlorophenyl)(1H-1,2,4-triazol-1-yl)methyl]-3-methyl-1,3-benzoxazol-2(3H)-one (33). F.° C. 70-74° C.

5-[(4-Cyanophenyl)(1H-1,2,4-triazol-1-yl)methyl]-6-methoxy-1,3-benzoxazol-2(3H)-one (34). F.° C. 125-130° C.

6-[1H-Imidazol-1-yl(phenyl)methyl]-1,3-benzothiazol-2(3H)-one (35).F ° C. 55-60° C

6-[1H-Imidazol-1-yl(phenyl)methyl]-3-methyl-1,3-benzothiazol-2(3H)-one (36). F.° C. 65-68° C

3-Methyl-6-[phenyl(1H-1,2,4-triazol-1-yl)methyl]-1,3-benzothiazol-2(3H)-one (37). F.° C. 150-154° C

6-[(4-Chlorophenyl)(1H-1,2,4-triazol-1-yl)methyl]-1,3-benzothiazol-2(3H)-one (38). F.° C. 106-112° C.

6-[1H-Imidazol-1-yl(4-nitrophenyl)methyl]-1,3-benzothiazol-2(3H)-one (39). F ° C. 238-241

4-Methyl-7-[1H-imidazol-1-yl(phenyl)methyl]-1,4-benzoxazin-3(4H)-one (40). F ° C. 66-68° C.

4-Methyl-7-[phenyl(1H-1,2,4-triazol-1-yl)methyl]-1,4-benzoxazin-3(4H)-one (41). F ° C. 160-164° C.

4-Methyl-6-[phenyl(1H-1,2,4-triazol-1-yl)methyl]-1,4-benzoxazin-3(4H)-one (42). F ° C. 140-150° C.

7-[1H-Imidazol-1-yl(phenyl)methyl]-1,4-benzothiazin-3(4H)-one (43). F ° C. 187-189° C.

PREPARATION OF COMPOUNDS OF EXAMPLES 44 to 49
(Tables I-B, III-B, IV)

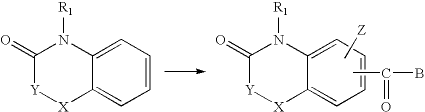

| Ref | R₁ | X | Y | Z | Isomer | B |
|---|---|---|---|---|---|---|
| 1 | H | S | — | H | 6 | 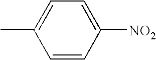 |
| 2 | CH₂CH₃ | S | — | H | 6 | 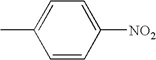 |
| 3 | H | Se | — | H | 6 | 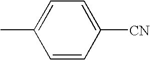 |
| 4 | CH₃ | Se | — | H | 6 | 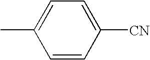 |
| 5 | CH₂CH₃ | Se | — | H | 6 | 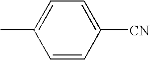 |
| 6 | H | Se | — | H | 6 | 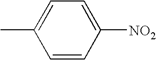 |
| 7 | CH₃ | Se | — | H | 6 | 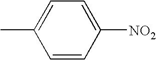 |
| 8 | CH₂CH₃ | Se | — | H | 6 | 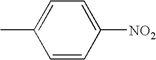 |

6-(4-Nitrobenzoyl)-1,3-benzothiazol-2(3H)-one (1; Table I-B). In a 100 ml flask containing 35.0 g (265 mmol) aluminium chloride, add drop by drop and with magnetic stirring 5.9 ml of dimethylformamide (76 mmol). Continue stirring for 25 minutes, slowly add 5.0 g (33 mmol) of 2(3H)-benzothiazolone and heat to 90° C. Add drop by drop 7.36 g of 4-nitrobenzoyl chloride (40 mmol) and continue to stir at 100-110° C. for 4 hours. Slowly pour the reaction mixture onto ice while stirring vigorously. Add 15 ml of 37% hydrochloric acid and then stir for 15 minutes. Spin out the precipitate then wash with water until the wash water is neutral. Dry the product obtained and recrystallise it in dioxane (5.85 g, 59%). Rf=0.39 (EtOAc/Cyclohexane=4/6): mp 260-265° C.; ir γ NH 3369 cm⁻¹, CO 1682 cm⁻¹, 1651 cm⁻¹, NO₂ 1521 cm⁻¹; ¹H-NMR (300 MHz, DMSO-d₆) δ 7.26 (d, 1H, H₄, J₄₋₅=7.8 Hz), 7.72-7.74 (m, 1H, H₅), 7.92 (d, 2H, H_{3′}, H_{5′}, J=9.0 Hz), 8.09 (s, 1H, H₇), 8.36 (d, 2H, H_{2′}, H_{6′}, J=9.0 Hz), 12.3 (br s, 1H, NH, exchangeable with D₂O). Anal. (C₁₄H₈N₂O₄S)
3-Ethyl-6-(4-nitrobenzoyl)-1,3-benzothiazol-2(3H)-one (2). In a 100 ml round-bottom flask, dissolve 2.5 g (8.3 mmol) of 6-(4-nitrobenzoyl)-1,3-benzothiazol-2(3H)-one in 25 ml of acetone. Add 3.5 g (25 mmol) of potassium carbonate and heat to 60° C. for 1 hour. Add drop by drop and with magnetic stirring 0.08 ml (10 nmol) of iodoethane. Stir at ambient temperature for 6 hours. The reaction mixture acetone is evaporated.
Add 70 ml of water and 6 N HCl until an acid pH is reached. Spin out the precipitate formed, wash with water, dry it and recrystallise it with acetonitrile (2.33 g, 85%). Rf=0.69 (EtOAc/Cyclohexane=5/5): mp 148-152° C.; ir γ CO 1678 cm⁻¹, 1622 cm⁻¹, NO₂ 1518 cm⁻¹; ¹H-NMR (300 MHz, DMSO-d₆) δ 1.20 (t, 3H, CH₃), 4.00 (q, 2H, CH₂), 7.54 (d, 1H, H₄, J₄₋₅=8.1 Hz), 7.77 (dd, 1H, H₅, J₅₋₄=8.1 Hz, J₅₋₇=1.8 Hz), 7.93 (d, 2H, H_{3′}, H_{5′}, J=9 Hz), 8.17 (d, 1H, H₇, J₇₋₅=1.8 Hz), 8.35 (d, 2H, H_{2′}, H_{6′}, J=9 Hz). Anal. (C₁₆H₁₂N₂O₄S)
4-[(2-Oxo-2,3-dihydro-1,3-benzoselenazol-6-yl)carbonyl]benzonitrile (3). It is identical to that described for the obtaining of (1) page 2. 2(3H)-benzoselenazolone (5 g, 25 mmol), dimethylformamide (4.5 ml, 58 mmol), aluminium chloride (26.9 g, 202 mmol) and 4-cyanobenzoyl chloride (6.58 g, 30 mmol), the product 3 obtained and recrystallised in acetonitrile (4.1 g, 50%). Rf=0.41 (EtOAc/Cyclohexane=4/6): mp 230-232° C.; ir γ NH 3248 cm⁻¹, CN 2229 cm⁻¹, CO 1701 cm⁻¹, 1678 cm⁻¹ 1H-NMR(300 MHz,DMSO-d₆) δ 7.22 (d, 1H, H₄, J₄₋₅=9.0 Hz), 7.67-7.70 (m, 1H, H₅), 7.82 (d, 2H, H₃, H_{5′}, J=8.1 Hz), 8.00 (d, 2H, H₂, H₆ J=8.1 Hz), 8.16 (s, 1H, H₇), 12.18 (br s, 1H, NH, exchangeable with D₂O). Anal. (C₁₅H₁₈N₂O₂Se)
4-[(2-Methyl-2-oxo-2,3-dihydro-1,3-benzoselenazol-6-yl)carbonyl]benzonitrile (4). It is identical to that described for the obtaining of (1) page 2. 3-methyl-2(3H)-benzoselenazolone (5 g, 24 mmol), dimethylformamide (4.2 ml, 54 mmol), aluminium chloride (25 g, 189 mmol) and 4-cyanobenzoyl chloride (4.7 g, 28 mmol), the product 4 obtained and recrystallised in acetonitrile (6.4 g, 80%). Rf=0.51 (EtOAc/Cyclohexane=4/6): mp 205-210° C.; ir γ CN 2231 cm⁻¹, CO 1699 cm⁻¹, 1658 cm⁻¹; ¹H-NMR (300 MHz, DMSO-d₆) δ 3.45 (s, 3H, CH₃), 7.45 (d, 1H, H₄, J₄₋₅=8.1 Hz), 7.76-7.78 (m, 1H, H₅), 7.83 (d, 2H, H_{3′}, H_{5′}, J=8.1 Hz), 8.02 (d, 2H, H_{2′}, H_{6′}, J=8.1 Hz), 8.25 (s, 1H, H₇). Anal. (C₁₆H₁₀N₂O₂Se)
4-[(3-Ethyl-2-oxo-2,3-dihydro-1,3-benzoselenazol-6-yl) carbonyl]benzonitrile (5). It is identical to that described for the obtaining of (2) page 2. 4-[(2-oxo-2,3-dihydro-1,3-benzoselenazol-6-yl)carbonyl]benzonitrile (1.2 g, 3.7 mmol), acetone (50 ml), potassium carbonate (1.52 g, 11 mmol) and iodoethane (0.35 ml, 4.4 mmol), the product 5 obtained and recrystallised in acetonitrile (1.1 g, 87%). Rf=0.55 (EtOAc/Cyclohexane=4/6): mp 130-135° C.; ir γ CN 2231 cm⁻¹, CO 1697 cm⁻¹, 1674 cm⁻¹; ¹H-NMR (300 MHz, DMSO-d₆) δ 1.19 (t, 3H, CH₃), 4.00 (q, 2H, CH₂), 7.50 (d, 1H, H₄, J₄₋₅=8.4 Hz), 7.76 (dd, 1H, H₅, J₅₋₄=8.4 Hz, J₅₋₇=1.5 Hz), 7.85 (d, 2H, H_{3′}, H_{5′}, J=8.4 Hz), 8.02(d, 2H, H_{2′}, H_{6′}, J=8.4 Hz), 8.27(s, 1H, H₇). Anal. (C₁₇H₁₂N₂O₂Se)
6-(4-Nitrobenzoyl)-1,3-benzoselenazol-2(3H)-one (6). It is identical to that described for the obtaining of (1) page 2. 3-methyl-2(3H)-benzoselenazolone (5 g, 24 mmol), dimethylformamide (4.2 ml, 54 mmol), aluminium chloride (25 g, 189 mmol) and 4-nitrobenzoyl chloride (5.62 g, 30 mmol), the product 6 obtained and recrystallised in acetonitrile (6.2 g, 70%). Rf=0.45 (EtOAc/Cyclohexane=4/6): mp 241-245° C.; ir γ NH 3250 cm⁻¹, CO 1695 cm⁻¹, 1647 cm⁻¹, NO₂ 1520 cm⁻¹; ¹H-NMR (300 MHz, DMSO-d₆) δ 7.25 (d, 1H, H₄, J₄₋₅=8.4 Hz), 7.70 (dd, 1H, H₅), J₅₋₄=8.4 Hz, J_{5-7′}=1.5 Hz, 7.91 (d, 2H, H_{3′}, H_{5′}, J=9.0 Hz), 8.18 (d, 1H, H₇, J₇₋₅=1.5 Hz), 8.35 (d, 2H, H₂, H₆, J=9.0 Hz), 12.2 (br s, 1H, NH, exchangeable with D₂O). Anal. (C₁₄H₈N₂O₄Se)
3-Methyl-6-(4-nitrophenyl)-1,3-benzoselenazol-2(3H)-one (7). It is identical to that described for the obtaining of (2) page 2. 6-(4-Nitrobenzoyl)-1,3-benzoselenazol-2(3H)-one (2.5 g, 7.2 mmol), acetone (100 ml), potassium carbonate (2.99 g, 22 mmol) and iodomethane (0.54 ml, 8.6 mmol), the product 7 obtained and recrystallised in acetonitrile (2.42 g, 93%). Rf=0.37 (EtOAc/Cyclohexane=3/7): mp 151-155° C.; ir γ CO 1680 cm⁻¹, 1655 cm⁻¹, NO₂ 1520 cm⁻¹; ¹H-NMR (300 MHz, DMSO-d₆) δ 3.45 (s, 3H, CH₃), 7.44 (d, 1H, H₄, J=8.7 Hz), 7.78 (dd, 1H, H₅, J₅₋₄=8.7 Hz, J₅₋₇=1.8 Hz), 7.92 (d, 2H, H_{3′}, H_{5′}, J=9.0 Hz), 8.28 (d, 1H, H₇, J₇₋₅=1.8 Hz), 8.36 (d, 2H, H_{2′}, H_{6′}, J=9.0 Hz). Anal. (C₁₅H₁₀N₂O₄Se)
3-Ethyl-6-(4-nitrobenzoyl)-1,3-benzoselenazol-2(3H)-one (8). It is identical to that described for the obtaining of (2) page 2. 6-(4-Nitrobenzoyl)-1,3-benzoselenazol-2(3H)-one (2.5 g, 7.2 mmol), acetone (100 ml), potassium carbonate (2.99 g, 22 mmol) and iodoethane (0.69 ml, 8.6 mmol), the product 8 obtained and recrystallised in methanol (2.2 g, 82%). Rf=0.60 (EtOAc/Cyclohexane=4/6): mp 97-102° C.; ir γ CO 1678 cm⁻¹, 1657 cm⁻¹, NO₂ 1520 cm⁻¹; ¹H-NMR (300 MHz, DMSO-d₆) δ 1.20 (t, 3H, CH₃), 4.01 (q, 2H, CH₂), 7.51 (d, 1H, H₄, J₄₋₅=8.4 Hz), 7.78 (dd, 1H, H₅, J₅₋₄=8.4 Hz, J₅₋₇=1.5 Hz), 7.94 (d, 2H, H_{3′}, H_{5′}, J=8.7 Hz), 8.30 (d, 1H, H₇, J₇₋₅=1.5 Hz), 8.37 (d, 2H, H_{2′}, H_{6′}, J=8.7 Hz). Anal. (C₁₆H₁₂N₂O₄Se)

### Reduction (Table III-B)

Reduction (Table III-B)

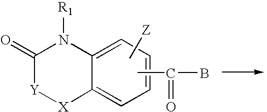

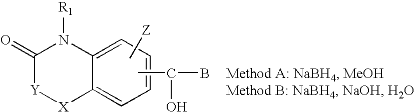

| | | | | | Iso- | | |
|---|---|---|---|---|---|---|---|
| Ref | R₁ | X | Y | Z | mer | B | Method |
| 1a | CH₂CH₃ | S | — | H | 6 | 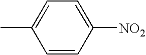 | A |
| 2a | H | Se | — | H | 6 | 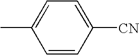 | A |
| 3a | CH₃ | Se | — | H | 6 | 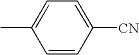 | A |
| 4a | CH₂CH₃ | Se | — | H | 6 | 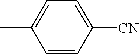 | A |
| 5a | CH₃ | Se | — | H | 6 | 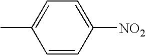 | A |
| 6a | CH₂CH₃ | Se | — | H | 6 | 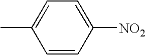 | A |

3-Ethyl-6-[hydroxy(4-nitrophenyl)methyl]-1,3-benzothiazol-2(3H)-one (1a). In a 100 ml round-bottom flask containing 2.3 g (7 mmol) of 3-ethyl-6-(4-nitrobenzoyl)-1,3-benzothiazol-2(3H)-one (2.3 g, 7 mmol), add 30 ml of methanol. Then, add little by little and with magnetic stirring, 0.3 g (7 mmol) of sodium borohydride. Continue stirring for 2 hours at ambient temperature. Evaporate all the solvent in a rotary evaporator, then take up the residue in 50 ml of slightly acid water. Spin out the precipitate formed, wash with water, until the wash water is neutral. Dry the product obtained and recrystallise it in ethyl acetate (2.2 g, 96%). Rf=0.4 (EtOAc/Cyclohexane=5/5); mp 160-162° C.; ir γ OH 3423 cm⁻¹, CO 1647 cm⁻¹, NO₂ 1520 cm⁻¹; ¹H NMR (300 MHz, DMSO-d₆) δ 1.14 (t, 3H, CH₃), 3.90 (q, 2H, CH₂), 5.89 (s, 1H, CH), 6.30 (s, 1H, OH, exchangeable with D₂O), 7.30 (d, 1H, H₄, J₄₋₅=8.1 Hz), 7.37-7.40 (m, 1H, H₅), 7.66-7.68 (m, 3H, H₇, H_{3′}, H_{5′}), 8.16 (d, 2H, H_{2′}, H_{6′}, J=8.1 Hz). Anal. (C₁₆H₁₄N₂O₄S)
4-[Hydroxy(2-oxo-2,3-dihydro-1,3-benzoselenazol-6-yl)methyl]benzonitrile (2a). It is identical to that described for the obtaining of (1a) page 4. 4-[(2-oxo-2,3-dihydro-1,3-benzoselenazol-6-yl)carbonyl]benzonitrile (2 g, 6.1 mmol), methanol (30 ml) and sodium borohydride (0.5 g, 6.1 mmol), the product 2a obtained and recrystallised in acetonitrile. (1.4 g, 70%). Rf=0.37 (EtOAc/Cyclohexane=5/5): mp 209-213° C.; ir γ OH 3506 cm⁻¹, NH 3146 cm⁻¹, CN 2227 cm⁻¹, CO 1695 cm⁻¹; ¹H-NMR (300 MHz, DMSO-d₆) δ 5.76 (s, 1H, CH), 6.17(s, 1H, OH, exchangeable with D₂O), 7.02 (d, 1H, H₄, J₄₋₅=8.1 Hz), 7.25 (dd, 1H, H₅, J₅₋₄=8.1 Hz, J₅₋₇=1.5 Hz), 7.54 (d, 3H, H_{3′}, H_{5′}, J=8.1 Hz), 7.66 (d, 2H, H_{2′}, H_{6′}, J=8.1 Hz), 7.43 (d, 1H, H₇, J₇₋₅=1.5 Hz), 11.85 (br s, 1H, NH, exchangeable with D₂O). Anal. (C₁₅H₁₀N₂O₂Se)
4-[Hydroxy(3-methyl-2-oxo-2,3-dihydro-1,3-benzoselenazol-6-yl)methyl]benzonitrile (3a). It is identical to that described for the obtaining of (1a) page 4. 4-[(3-Methyl-2-oxo-2,3-dihydro-1,3-benzoselenazol-6-yl)carbonyl]benzonitrile (2.0 g, 5.9 mmol), methanol (50 ml) and sodium borohydride (1.2 g, 32 mmol), the product 3a obtained and recrystallised in ethyl acetate (1.8 g, 90%). Rf=0.38 (EtOAc/Cyclohexane=5/5); mp 205-208° C.; ir γ OH 3472 cm⁻¹, CN 2224 cm⁻¹, CO 1651 cm⁻¹; ¹H-NMR (300 MHz, DMSO-d₆) δ 3.30 (s, 3H, CH₃), 5.80 (s, 1H, CH), 5.82 (s, 1H, OH, exchangeable with D₂O), 7.19 (d, 1H, H₄, J₄₋₅=8.4 Hz), 7.34-7.36 (m, 1H, H₅), 7.55 (d, 2H, H_{3′}, H_{5′}, J=7.8 Hz), 7.73-7.77 (m, 3H, H₇, H_{2′}, H_{6′}). Anal. (C₁₆H₁₂N₂O₂Se)
4-[(3-Ethyl-2-oxo-2,3-dihydro-1,3-benzoselenazol-6-yl)(hydroxy) methyl]benzonitrile (4a).
It is identical to that described for the obtaining of (1a) page 4. 4-[(3-ethyl-2-oxo-2,3-dihydro-1,3-benzoselenazol-6-yl)carbonyl]benzonitrile (1.1 g, 3.0 mmol), methanol (15 ml) and sodium borohydride (0.06 g, 1.5 mmol), the product 4a obtained and recrystallised in ethyl acetate (0.92 g, 86%). Rf=0.31 (EtOAc/Cyclohexane=4/6): mp 132-134° C.; ir γ OH 3427 cm⁻¹, CN 2227 cm⁻¹, CO 1641 cm⁻¹; ¹H-NMR (300 MHz, DMSO-d₆) δ 1.13 (t, 3H, CH₃), 3.89 (q, 2H, CH₂), 5.80 (d, 1H, CH, J=3.9 Hz), 6.19 (d, 1H, OH, J=3.6 Hz, exchangeable with D₂O), 7.26 (d, 1H, H₄, J₄₋₅=8.1 Hz), 7.34 (dd, 1H, H₅, J₅₋₄=8.1 Hz, J₅₋₇=1.8 Hz), 7.57 (d, 2H, H_{3′}, H_{5′}, J=8.4 Hz), 7.75-7.79 (m, 3H, H₇, H_{2′}, H_{6′}). Anal. (C₁₇H₁₄N₂O₂Se)
6-[Hydroxy(4-nitrophenyl)methyl]-3-methyl-1,3-benzoselenazol-2(3H)-one (5a). It is identical to that described for the obtaining of (1a) page 4. 3-Methyl-6-(4-nitrophenyl)-1,3-benzoselenazol-2(3H)-one (2.3 g, 6.4 mmol), methanol (30 ml) sodium borohydride (0.3 g, 6.4 mmol), the product 5a obtained and recrystallised in acetonitrile (1.9 g, 84%). Rf=0.31 (EtOAc/Cyclohexane=4/6); mp 182-183° C.; ir γ OH 3406 cm¹, CO is 1645 cm⁻¹, NO₂ 1512 cm⁻¹; ¹H NMR (300 MHz, DMSO-d₆) δ 3.35 (s, 3H, CH₃), 5.88 (s, 1H, CH), 6.29 (s, 1H, OH, exchangeable with D₂O), 7.21 (d, 1H, H₄, J₄₋₅=8.1 Hz), 7.37 (dd, 1H, H₅, J₅₋₄=8.1 Hz, J₅₋₇=1.8 Hz), 7.64 (d, 2H, H_{3′}, H_{5′}, J=8.7 Hz), 7.75 (d, 1H, H₇, J₇₋₅=1.8 Hz), 8.16 (d, 2H, H_{2′}, H_{6′}, J=8.7 Hz). Anal. (C₁₅H₁₂N₂O₄Se)
3-Ethyl-6-[hydroxy(4-nitrophenyl)methyl]-1,3-benzoselenazol-2(3H)-one (6a). It is identical to that described for the obtaining of (1a) page 4. 3-Ethyl-6-(4-nitrobenzoyl)-1,3-benzoselenazol-2(3H)-one (2.2 g, 5.8 mmol), methanol (30 ml) sodium borohydride (0.3 g, 5.8 mmol), the product 6a obtained and recrystallised in ethyl acetate (1.2 g, 57%). Rf=0.35 (EtOAc/Cyclohexane=4/6); mp 135-137° C.; ir γ OH 3420 cm⁻¹, CO 1653 cm⁻¹, NO₂ 1514 cm⁻¹; ¹H NMR (300 MHz, DMSO-d₆) δ 1.13 (t, 3H, CH₃), 3.89 (q, 2H, CH₂), 5.87 (s, 1H, CH), 6.28 (s, 1H, OH, exchangeable with D₂O), 7.27 (d, 1H, H₄, J₄₋₅=8.4 Hz), 7.36 (dd, 1H, H₅, J₅₋₄=8.4 Hz, J₅₋₇=1.8 Hz), 7.65(d, 2H, H_{3′}, H_{5′}, J=9 Hz), 7.76 (d, 1H, H₇, J₇₋₅=1.8 Hz), 8.17-8.20 (m, 2H, H_{2′}, H_{6′}). Anal. (C₁₆H₁₄N₂O₄Se)

Substitution

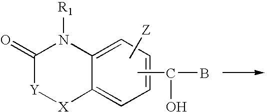

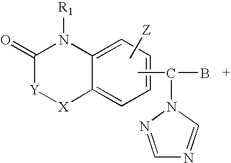

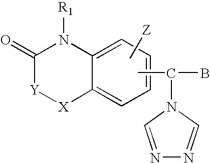

| | | | | | Iso- | | |
|---|---|---|---|---|---|---|---|
| Ref | R₁ | X | Y | Z | mère | triazole | B |
| 1b | CH₂CH₃ | S | — | H | 6 | 1,2,4 | 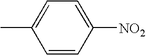 |
| 2b | H | Se | — | H | 6 | 1,2,4 | 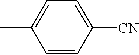 |
| 3b | CH₃ | Se | — | H | 6 | 1,2,4 | 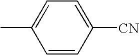 |
| 4b | CH₂CH₃ | Se | — | H | 6 | 1,2,4 | 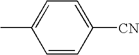 |
| 5b | CH₃ | Se | — | H | 6 | 1,2,4 | 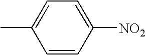 |
| 6b | CH₂CH₃ | Se | — | H | 6 | 1,2,4 | 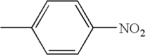 |

### Example 44

### 3-Ethyl-6-[(4-nitrophenyl)(1H-1,2,4-triazol-1-yl)methyl]-1,3-benzothiazol-2(3H)-one

In a 100 ml round-bottom flask, dissolve 4.83 g (70 mmol) of 1H-1,2,4-triazol in 35 ml of acetonitrile then slowly add 1.3 ml (18 mmol) of thionyl chloride. Continue stirring for 30 minutes at ambient temperature. Collect the filtrate obtained. The filtrate is added drop by drop to a solution of 1.5 g (4.5 mmol) of 3-ethyl-6-[hydroxy(4-nitrophenyl)methyl]-1,3-benzothiazol-2(3H)-one and 10 ml of acetonitrile. Continue stirring for 5 hours at ambient temperature. Evaporate the solvent in a rotary evaporator: Add 100 ml of water and add 6 N HCl until an acid pH is reached. Extract with 150 ml of ethyl acetate. The aqueous phase is alkalized with a solution of potassium carbonate as far as neutral. Extract with 150 ml of ethyl acetate, dry the organic phase on MgSO₄ then vaporize it and purify it by chromatography on silica gel. (eluant: EtOAc) (0.34 g, 20%). Rf=0.28 (EtOAc): mp 79-83° C.; ir γ CO 1676 cm⁻¹, 1602 cm⁻¹, NO₂ 1520 cm⁻¹; ¹H-NMR (300 MHz, CDCl₃) δ 1.17 (t, 3H, CH₃), 3.93 (q, 2H, CH₂), 7.30-7.35 (m, 2H, CH, H₄), 7.40-7.47 (m, 3H, H₅, H_{3′}, H_{5′}), 7.62 (s, 1H, H₇), 8.12 (s, 1H, H_{triazole}), 8.23 (d, 2H, H_{2′}, H_{6′}, J=8.1 Hz), 8.69 (s, 1H, H_{triazole}). Anal. (C₁₇H₁₃N₅O₃S)

### Example 45

### 4-[(2-oxo-2,3-dihydro-1,3-benzoselenazol-6-yl)(1H-1,2,4-triazol-1-yl)methyl]benzonitrile

It is identical to that described for the obtaining is of (1b) page 6. 4-[Hydroxy(2-oxo-2,3-dihydro-1,3-benzoselenazol-6-yl)methyl]benzonitrile (1.5 g, 4.6 mmol), thionyl chloride (1.3 ml, 18 mmol), 1H-1,2,4-triazol (4.84 g, 70 mmol) and THF (35 ml), the product 2b obtained and purified by silica gel chromatography (eluant: EtOAc) (0.17 g, 10%). Rf=0.46 (EtOAc): mp 223-226° C.; ir γ NH 3435 cm⁻¹, CN 2229 cm⁻¹, CO 1685 cm⁻¹; ¹H-NMR (300 MHz, DMSO-d₆) δ 7.09 (d, 1H, H₄, J₄₋₅=8.1 Hz), 7.13 (dd, 1H, H₅, J₅₋₄=8.1 Hz, J₅₋₇=1.5 Hz), 7.20 (s, 1H, CH), 7.33 (d, 2H, H_{3′}, H_{5′}, J=7.8 Hz), 7.56 (d, 1H, H₇, J₇₋₅=1.5 Hz), 7.83 (d, 2H, H_{2′}, H_{6′}, J=7.8 Hz), 8.08 (s, 1H, H_{triazole}), 8.62 (s, 1H, H_{triazole}), 11.83 (br s, 1H, NH, exchangeable with D₂O). Anal. (C₁₇H₁₁N₅OSe)

### Example 46

### 4-[(3-Methyl-2-oxo-2,3-dihydro-1,3-benzoselenazol-6-yl)(1H-1,2,4-triazol-1-yl)methyl]benzonitrile

It is identical to that described for the obtaining of (1 b) page 6. 4-[Hydroxy(3-methyl-2-oxo-2,3-dihydro-1,3-benzoselenazol-6-yl)methyl]benzonitrile (1.5 g, 4.4 mmol), thionyl chloride (1.3 ml, 18 mmol), 1H-1,2,4-triazol (4.65 g, 67 mmol) and acetonitrile (40 ml), the product 2b obtained and purified by silica gel chromatography (eluant: EtOAc) (0.35 g, 20%). Rf=0.42 (EtOAc): mp 154-158° C.; ir γ CN 2229 cm⁻¹ CO 1657 cm⁻¹; ¹H-NMR (300 MHz, DMSO-d₆) δ 3.37 (s, 3H, CH₃), 7.25-7.30 (m, 3H, CH, H₄, H₅), 7.34 (d, 2H, H_{3′}, H_{5′}, J=8.7 Hz), 7.66 (s, 1H, H₇), 7.84 (d, 2H, H_{2′}, H_{6′}, J=8.7 Hz), 8.09 (s, 1H. H_{triazole}), 8.64 (s, 1H. H_{triazole}). Anal. (C₁₈H₁₃N₅OSe)

### Example 47

### 4-[(3-Ethyl-2-oxo-2,3-dihydro-1,3-benzoselenazol-6-yl)(1H-1,2,4-triazol-1-yl)methyl]benzonitrile

It is identical to that described for the obtaining of (1 b) page 6. 4-[(3-Ethyl-2-oxo-2,3-dihydro-1,3-benzoselenazol-6-yl)(hydroxy)methyl]benzonitrile (0.9 g, 2.5 mmol), thionyl chloride (0.7 ml, 10 mmol), 1H-1,2,4-triazol (2.68 g, 39 mmol) and acetonitrile (35 ml), the product 2b obtained and purified by silica gel chromatography (eluant: EtOAc) (0.2 g, 19%). Rf=0.44 (EtOAc); mp 95-98° C.; ir γ CN 2229 cm⁻¹, CO 1670 cm⁻¹; ¹H NMR (300 MHz, DMSO-d₆) δ 1.15 (t, 3H, CH₃), 3.91 (q, 2H, CH₂), 7.26 (m, 2H, CH, H₄), 7.35-7.39 (m, 3H, H₅, H_{3′}, H_{5′}), 7.69 (s, 1H, H₇), 7.86 (d, 2H, H_{2′}, H_{6′}, J=8.1 Hz), 8.11 (s, 1H. H_{triazole}), 8.67 (s, 1H. H_{triazole}). Anal. (C₁₉H₁₅N₅OSe)

### Example 48

### 3-Methyl-6-[(4-nitrophenyl)(1H-1,2,4-triazol-1-yl)methyl]-1,3-benzoselenazol-2(3H)-one

It is identical to that described for the obtaining of (1b) page 6. 6-[Hydroxy(4-nitrophenyl)methyl]-3-methyl-1,3-benzoselenazol-2(3H)-one (1.5 g, 4.1 mmol), thionyl chloride (1.γ ml, 17 mmol), 1H-1,2,4-triazol (4.39 g, 64 mmol) and acetonitrile (40 ml), the product 2b obtained and purified by silica gel chromatography (eluant: EtOAc) (0.29 g, 17%). Rf=0.46 (EtOAc); mp 190-195° C.; ir γ CO 1651 cm⁻¹, NO₂ 1520 cm⁻¹; ¹H NMR (300 MHz, DMSO-d₆) δ 3.36 (s, 3H, CH₃), 7.30-7.35 (m, 3H, CH, H₄, H₅), 7.44 (d, 2H, H_{3′}, H_{5′}, J=8.7), 7.69 (s, 1H, H₇), 8.12 (s, 1H, H_{triazole}), 8.24 (d, 2H, H_{2′}, H_{6′}, J=8.7), 8.68 (s, 1H, H_{triazole}). Anal. (C₁₇H₁₃N₅O₃Se)

### Example 49

### 3-Ethyl-6-[(4-nitrophenyl)(1H-1,2,4-triazol-1-yl)methyl]-1,3-benzoselenazol-2(3H)-one

It is identical to that described for the obtaining of (1b) page 6. 3-ethyl-6-[Hydroxy(4-nitrophenyl)methyl]-1,3-benzoselenazol-2(3H)-one (1.2 g, 3.2 mmol), thionyl chloride (0.9 ml, 13 mmol), 1H-1,2,4-triazol (3.38 g, 49 mmol) and acetonitrile (35 ml), the product 2b obtained and purified by silica gel chromatography (eluant: EtOAc) (0.28 g, 21%). Rf=0.44 (EtOAc): mp 79-82° C; ir γ CO 1670 cm⁻¹, NO₂ 1520 cm⁻¹; ¹H-NMR (300 MHz, CDCl₃) δ 1.13 (t, 3H, CH₃), 3.91 (q, 2H, CH₂), 7.27-7.39 (m, 3H, CH, H₄, H₅), 7.45 (d, 2H, H_{3′}, H_{5′}, J=8.7 Hz), 7.70 (s, 1H, H₇), 8.12 (s, 1H, H_{triazole}), 8.24 (d, 2H, H_{2′}, H_{6′}, J=8.7 Hz), 8.69 (s, 1H, H_{triazole}) Anal. (C₁₈H₁₅N₅O₃Se)

### Preparation of Compounds of Examples 50 to 51 (Tables I-B, III-B, IV)

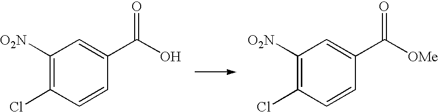 Methyl 4-chloro-3-nitrenzoate (1). Dissolve 4-chloro-3-nitro-benzoic acid (5.0 g, 24.8 mmol) in 200 ml of methanol and add 4.15 ml (29.8 mmol) of triethylamine. Cool in an ice-salt bath and add drop by drop 3.19 ml (44.7 mmol) of acetyl chloride. Agitate at reflux for 6 hours. Evaporate the solvent under reduced pressure. Take up the residue with 100 ml of water and extract 2 times with ethyl acetate (100 ml). Dry the organic phase on MgSO₄ and vaporize it under reduced pressure and purify it with ether (10 ml) (4.81 g, 92%). Rf=0.55 (EtOAc/Cyclohexane=7/3); mp 79-80° C.; ir CO 1716 cm⁻¹; ¹H-NMR(300 MHz, DMSO-d₆) δ 3.90 (s, 3H, OCH₃), 7.90 (d, 1H, H₅, J₅₋₆=8.1 Hz), 8.15 (dd, 1H, H₆, J₆₋₅=8.1 Hz, J₅₋₂=1.5 Hz), 8.49(d, 1H, H₂, J₂₋₆=1.5 Hz). Anal. (C₈H₆ClNO₄). 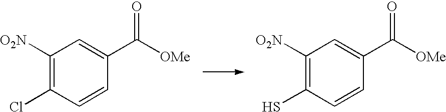
Methyl-3-nitro-4-sulfanylbenzoate (2). In a 250 ml round-bottom flask, put into suspension sodium sulphate (2.7 g, 34 mmol) and methyl-4-chloro-3-nitrobenzoate (5 g, 23 mmol) in 150 ml of absolute ethanol. Stir at ambient temperature for 7 hours. Pour the reaction mixture on ice (200 ml). Add acetic acid as far as pH 2 and extract 3 times with CH₂Cl₂ (100 ml). Dry the organic phase on MgSO₄ and vaporize it under reduced pressure and purify it with ether (3.9 g, 80%). Rf=0.31 (EtOAc/Cyclohexane=3/7); mp 98-101° C.; ir SH 2546, CO 1722 cm⁻¹; ¹H-NMR (300 MHz, DMSO-d₆) δ 3.81 (s, 3H, OCH₃), 4.31 (br s, 1H, SH, exchangeable with D₂O), 7.82 (d, 1H, H₅, J₅₋₆=8.2 Hz), 8.17 (dd, 1H, H₆, J₆₋₅=8.2 Hz, J₅₋₂=1.5 Hz), 8.41 (d, 1H, H₂, J₂₋₆=1.5 Hz). Anal. (C₈H₇NO₄S). 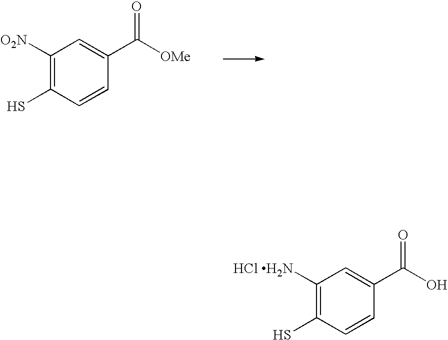
3-Amino-4-sulfanyl benzoic acid hydrochloride (3). In a 250 ml round-bottom flask, put into suspension tin (II) chloride (17.3 g, 91.4 mmol) and methyl-3-nitro-4-sulfanylbenzoate (3.9 g, 18.3 mmol) in 50 ml of 6 N HCl. Agitate by reflux for 4 hours. Pour the reaction mixture on ice (200 ml). Spin out the precipitate formed, dry it and recrystallise it with ether (3.3 g, 81%). Rf=0.32 (EtOAc/Cyclohexane=5/5); mp 215-217° C. (decomposition); ir NH₂ 3331 cm⁻¹, SH 2511 cm⁻¹, CO 1711 cm⁻¹; ¹H-NMR (300 MHz, DMSO-d₆) δ 4.42 (br s, 1H, SH, exchangeable with D₂O), 7.76 (d, 1H, H₅, J₅₋₆=8.2 Hz), 8.31 (dd, 1H, H₆, J₆₋₅=8.1 Hz, J₅₋₂=1.5 Hz), 8.44(d, 1H, H₂, J₂₋₆=1.5 Hz), 12.2 (br s, 1H, OH, exchangeable with D₂O). Anal. (C₈H₁₀NO₂ClS). 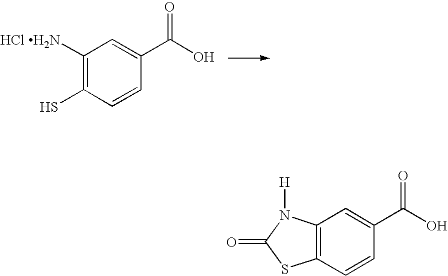
2-Oxo-2,3-dihydro-1,3-benzothiazolone-5-carboxylic acid (4). Mix 5 g (24.3 mmol) of 3-amino-4-sulfanyl benzoic acid HCl salt and 14.6 g (243 mmol) of urea. Stir at 140-145° C. for 4 hours. Pour the reaction is mixture on ice (200 ml) and add 6N acetic acid as far as pH 2. Spin out the precipitate formed, dry it and recrystallise it with ether (2.9 g, 49%). Rf=0.65 (MeOH/EtOH/Cyclohexane=3/5/2), mp 275-277° C.; ir OH 3099 cm⁻¹, CO 1718 cm⁻¹, NCO 1682 cm⁻¹; ¹H-NMR (300 MHz, DMSO-d₆) δ 7.62 (s, 1H, H₄), 7.69-7.72 (m, 2H, H_{5,6}), 12.10(br s, 1H, NH, exchangeable with D₂O), 13.06 (br s, 1H, exchangeable with D₂O). Anal. (C₉H₇NO₃S). 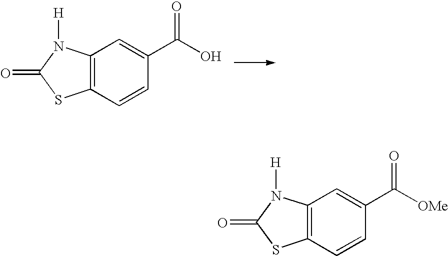
Methyl-2-oxo-2,3-benzothiazolone-5-carboxylate (5). Put the 2-oxo-2,3-dihydro-1,3-benzothiazolone-5-carboxylic acid (5.0 g, 24.8 mmol) in 200 ml of methanol. Cool in an ice-salt bath at 0° C. and add drop by drop 9.34 ml (128.1 mmol) of thionyl chloride. Agitate by reflux for 5 hours. Evaporate the solvent under reduced pressure. Take up the residue with 100 ml of water and extract 2 times with ethyl acetate (100 ml). Dry the organic phase on MgSO₄ and vaporize it under reduced pressure and purify it with ether (10 ml) (4.0 g, 75%). Rf=0.58 (EtOAc/Cyclohexane=5/5); mp 217-219° C; ir CO 1695 cm⁻¹, NCO 1684 cm⁻¹; ¹H-NMR(300 MHz, DMSO-d₆) δ 3.85 (s, 3H, OCH₃), 7.60 (d, 1H, H₄, J₄₋₆=2.7 Hz), 7.67-7.69 (m, 2H, H_{6,7}), 12.13 (br s, 1H, NH, exchangeable with D₂O). Anal. (C₉H₇NO₃S). 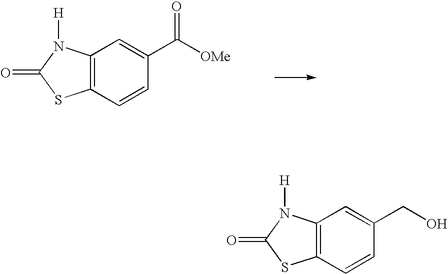
5-(Hydroxymethyl)-1,3-benzothiazol-2(3H)-one (6). Dissolve the methyl-2-oxo-2,3-benzothiazolone-5-carboxylate (5.0 g, 23.9 mmol) in is 1100 ml of THF. Cool in an ice-salt bath and add little by little 1.1 g (28.7 mmol) of LiAlH₄. Stir at ambient temperature for 3 hours. Slowly add 100 ml of water to the reaction mixture and add 1 N acetic acid as far as pH 7. Extract 2 times with CH₂Cl₂ (100 ml). Dry the organic phase on MgSO₄ and vaporize it under reduced pressure and purify it with ether (10 ml) (3.4 g, 79%). Rf=0.33 (EtOAc/Cyclohexane=3/7); mp 178-181° C.; ir OH 3319 cm⁻¹, NCO 1684 cm⁻¹; ¹H-NMR(300 MHz, DMSO-d₆) δ 4.49 (d, 2H, CH₂OH, J=5.7 Hz), 5.26 (t, 1H, CH₂OH, J=5.7 Hz, exchangeable with D₂O), 7.02 (d, 1H, H₆, J₆₋₇=8.1 Hz), 7.09 (s, 1H, H₄), 7.45 (d, 1H, H₇, J₇₋₆=8.1 Hz), 11.85 (s, 1H, NH, exchangeable with D₂O). Anal. (C₈H₇NO₂S).

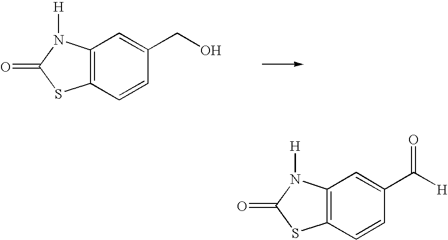

2-Oxo-2,3-dihydro-1,3-benzothiazol-5-carbaldehyde (7). Dissolve the 5-(hydroxymethyl)-1,3-benzothiazol-2(3H)-one (1 g, 5.5 mmol) in 100 ml of CH₂Cl₂. Add 10 g (177 mmol) manganese dioxide and stir at ambient temperature for 4 hours. Spin the reaction mixture and evaporate the solvent under reduced pressure and purify it with ether (10 ml) (0.69 g, 69%). Rf=0.56 (EtOAc/Cyclohexane=5/5); mp 211-215° C; ir CO 1730 cm⁻¹, NCO 1691 cm⁻¹; ¹H-NMR(300 MHz, DMSO-d₆) δ 7.53 (s, 1H, H₄), 7.65 (d, 1H, H₆, J₆₋₇=8.1 Hz), 7.80 (d, 1H, H₇, J₇₋₆=8.1 Hz), 9.95 (s, 1H, COH), 12.22 (br s, 1H, NH, exchangeable with D₂O). Anal. (C₈H₅NO₂S).

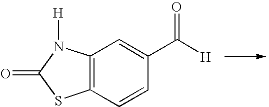

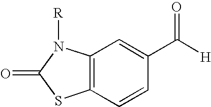

| Ref | R | yield |
|---|---|---|
| 8a | CH₃ | 84% |
| 8b | CH₂CH₃ | 87% |

3-Methyl-2-oxo-2,3-dihydro-1,3-benzothiazol-5-carbaldehyde (8a). In a 100 ml round-bottom flask, dissolve 1.0 g (5.6 mmol) of 2-oxo-2,3-dihydro-1,3-benzothiazol-5-carbaldehyde in 50 ml of acetone. Add 2.3 g (16.7 mmol) of potassium carbonate and 0.42 ml (6.7 mmol) of iodomethane. Stir at ambient temperature for 3 hours. The reaction mixture acetone is evaporated. Add 100 ml of water and extract 2 times with ethyl acetate (100 ml). Dry the organic phase on MgSO₄ and vaporize it under reduced pressure and purify it with ether (10 ml) (0.91 g, 84%). Rf=0.59 (EtOAc/Cyclohexane=5/5); mp 140-142° C.; ir CO 1682 cm⁻¹, NCO 1674 cm⁻¹; ¹H-NMR(300 MHz, DMSO-d₆) δ 3.46 (s, 3H, NCH₃), 7.73-7.75 (m, 2H, H_{4,6}), 7.90 (d, 1H, H₇, J₇₋₆=8.1 Hz), 9.99 (s, 1H, COH). Anal. (C₉H₇NO₂S).

3-Ethyl-2-oxo-2,3-dihydro-1,3-benzothiazol-5-carbaldehyde (8b). It is identical to that described to obtain (8a). 2-Oxo-2,3-dihydro-1,3-benzothiazol-5-carbaldehyde (2 g, 11.1 mmol), potassium carbonate (4.6 g, 33.3 mmol), iodoethane (1.1 ml, 13.3 mmol) and acetone (50 ml), the product 8b obtained and purified with ether (2.01 g, 87%). Rf=0.63 (EtOAc/Cyclo-hexane=5/5); mp 155-156° C.; ir CO 1689 cm⁻¹, NCO 1664 cm⁻¹; ¹H-NMR(300 MHz, DMSO-d₆) δ 1.23 (t, 3H, CH₂CH₃, J=6.7 Hz), 4.03 (q, 2H, CH₂CH₃, J=6.7 Hz), 7.74 (dd, 1H, H₆, J₆₋₇=8.1 Hz, J₆₋₄=2.1 Hz), 7.85 (d, 1H, H₄, J₄₋₆=2.1 Hz), 7.91 (d, 1H, H₇, J₇₋₆=8.1 Hz), 10.04 (s, 1H, COH). Anal. (C₁₀H₉NO₂S).

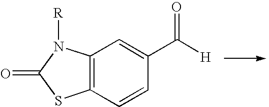

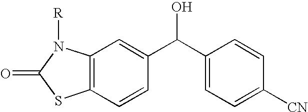

| Ref | R | yield |
|---|---|---|
| 9a | CH₃ | 18% |
| 9b | CH₂CH₃ | 29% |

4-[Hydroxy(3-methyl-2-oxo-2,3-dihydro-1,3-benzothiazol-5-yl)methyl]benzonitrile (9a).
Dissolve 4-bromobenzonitrile (1.9 g, 10.4 mmol) in 20 ml of THF and add 5.2 ml (10.4 mmol) of i-propyl magnesium chloride solution 2M in THF. Stir at ambient temperature for 2 hours. Next pour in drop by drop 2 g (10.4 mmol) of 3-methyl-2-oxo-2,3-dihydro-1,3-benzothiazol-5-carbaldehyde (2 g, 10.4 mmol) previously diluted in 20 ml of THF. Slowly add 100 ml of water into the reaction mixture and extract 2 times with ethyl acetate (100 ml). Dry the organic phase on MgSO₄ and vaporize it under reduced pressure and purify it by silica gel chromatography. (eluant: EtOAc/C-hexane=3/7) (0.55 g, 18%) Rf=0.29 (EtOAc/Cyclohexane=5/5); mp 183-186° C.; ir OH 3398 cm⁻¹, CN 2224 cm⁻¹, CO 1658 cm⁻¹; ¹H-NMR(300 MHz, DMSO-d₆) δ 3.38 (s, 3H, NCH₃), 5.84 (d, 1H, CH, J=3.9 Hz), 6.28 (d, 1H, OH, J=3.9 Hz, exchangeable with D₂O), 7.16 (d, 1H, H₇, J₇₋₆=8.1 Hz), 7.36 (s, 1H, H₄), 7.54 (d, 1H, H₆, J₆₋₇=8.1 Hz), 7.60 (d, 2H, H_{2′,6′}, J_{2′-3′}=8.1 Hz), 7.75 (d, 2H, H_{3′,5′}, J_{3′-2′}=8.1 Hz). Anal. (C₁₆H₁₂N₂O₂S).

4-[Hydroxy(3-ethyl-2-oxo-2,3-dihydro-1,3-benzothiazol-5-yl)methyl]benzonitrile (9b). It is identical to that described to obtain (9a). 3-Ethyl-2-oxo-2,3-dihydro-1,3-benzothiazol-5-carbaldehyde (2 g, 9.7 mmol), 4-bromobenzonitrile (1.7 g, 9.7 mmol), i-propyl magnesium chloride 2M solution in THF (4.8 ml, 9.7 mmol) and THF (40 ml), the is product 9b obtained and purified by silica gel chromatography (eluant: EtOAc/C-hexane=3/7) (0.87 g, 29%). Rf=0.31 (EtOAc/Cyclohexane=5/5); mp 156-158° C.; ir OH 3433 cm⁻¹, CN 2227 cm⁻¹, NCO 1674 cm⁻¹; ¹H-NMR(300 MHz, DMSO-d₆) δ 1.80 (t, 3H, CH₂CH₃, J=7.2 Hz), 3.93 (q, 2H, CH₂CH₃, J=7.2 Hz), 5.97 (d, 1H, CH, J=3.9 Hz), 6.30 (d, 1H, OH, J=3.9 Hz, exchangeable with D₂O), 7.17 (d, 1H, H₇, J₇₋₆=8.0 Hz), 7.45 (s, 1H, H₄), 7.56 (d, 1H, H₆, J₆₋₇=8.0 Hz), 7.62 (d, 2H, H_{2′,6′}, J_{2′-3′}32 8.1 Hz), 7.77 (d, 2H, H_{3′,5′}, J_{3′-2′}=8.1 Hz). Anal. (C₁₇H₁₄N₂O₂S).

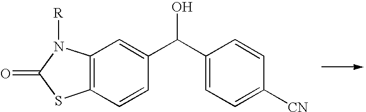

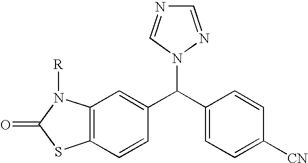

| Ref | R | yield |
|---|---|---|
| 10a | CH₃ | 32% |
| 10b | CH₂CH₃ | 21% |

### Example 50

### 4-[(3-Methyl-2-oxo-2,3-dihydro-1,3-benzothiazol-5-yl)(1H-1,2,4-triazol-1-yl)methyl]benzonitrile

In a 100 ml round-bottom flask, dissolve 1.3 g (18.8 mmol) of 1H-1,2,4-triazol in 20 ml of acetonitrile then slowly add 0.37 ml (5.1 mmol) of thionyl chloride. Continue stirring for 30 minutes at ambient temperature. Spin the filtrate obtained. The filtrate is added drop by drop to a solution of 0.38 g (1.3 mmol) of 4-[hydroxy(3-methyl-2-oxo-2,3-dihydro-1,3-benzothiazol-5-yl)methyl]benzonitrile and 10 ml of acetonitrile. Continue stirring for 5 hours at ambient temperature. Evaporate the solvent in a rotary evaporator. Add 100 ml of water and add 6 N HCl until an acid pH is reached. Extract with 150 ml of ethyl is acetate. The aqueous phase is alkalized with a solution of potassium carbonate as far as neutral. Extract with 150 ml of ethyl acetate, dry the organic phase on MgSO₄ then vaporise it and purify it by silica gel chromatography. (eluant: EtOAc/MeOH=9/1) (0.14 g, 32%). Rf=0.54 (EtOAc/MeOH=9/1): mp 122-125° C; ir CN 2229 cm⁻¹, NCO 1680 cm⁻¹; ¹H-NMR(300 MHz, DMSO-d₆) δ 3.34 (s, 3H, NCH₃), 7.10 (dd, 1H, H₆, J₆₋₇=8.1 Hz, J₆₋₄=1.5 Hz), 7.27-7.28 (m, 2H, CH, H₄), 7.35 (d, 2H, H_{2′,6′}, J_{2′-3′}=8.4 Hz), 7.66 (d, 1H, H₇, J₇₋₆=8.1 Hz), 7.84 (d, 2H, H_{3′,5′}, J_{3′-2′}=8.4 Hz), 8.11 (s, 1H, H_{triazole}), 8.66 (s, 1H, H_{triazole}). Anal. (C₁₈H₁₃N₅OS).

### Example 51

### 4-[(3-Ethyl-2-oxo-2,3-dihydro-1,3-benzothiazol-5-yl)(1H-1,2,4-triazol-1-yl)methyl]benzonitrile

It is identical to that described for the obtaining of (10a). 4-[Hydroxy(3-ethyl-2-oxo-2,3-dihydro-1,3-benzothiazol-5-yl)methyl]benzonitrile) (0.87 g, 2.8 mmol), 1,2,4-triazole (2.9 g, 42.0 mmol), thionyl chloride (0.82 ml, 1.1 mmol) and acetonitrile (100 ml), the product 2b obtained and purify it by silica gel chromatography (eluant: EtOAc/MeOH=9/1) (0.21 g, 21%). Rf=0.58 (EtOAc/MeOH=9/1); mp 125-127° C; ir CN 2229 cm⁻¹, NCO 1674 cm⁻¹; ¹H-NMR(300 MHz, DMSO-d₆) δ 1.12 (s, 3H, CH₂CH₃, J=7.5 Hz), 3.88 (q, 2H, CH₂CH₃, J=7.5 Hz), 7.10 (dd, 1H, H₆, J₆₋₇=8.1 Hz, J₆₋₄=1.5 Hz), 7.29 (s, 1H, CH), 7.35 (d, 2H, H_{2′,6′}, J_{2′-3′}=8.1 Hz), 7.40 (s, 1H, H₄), 7.68 (d, 1H, H₇, J₇₋₆=8.1 Hz), 7.86 (d, 2H, H_{2′,6′}, J_{2′-3′}=8.1 Hz), 8.12 (s, 1H, H_{triazole}), 8.69 (s, 1H, H_{triazole}). Anal. (C₁₉H₁₅N₅OS).

The examples above illustrate the invention and do not limit it in any way. The preparations above also comprise the intermediates of synthesis useful for the preparation of the compounds of formula (I) of the invention.

### Pharmacological Study (Table V)

### Example A

### Study of Acute Toxicity

The acute toxicity has been estimated after oral administration to groups of 8 mice (26 g). The animals were observed at regular intervals in the course of the first day and daily during the two weeks following treatment.

The dose at which 50% mortality in the animals (LD₅₀) is observed was measured and showed the low-level toxicity of the compounds of the invention.

### Example B

### Study of the Power of the Aromatase Inhibitor In Vitro

The IC₅₀, concentrations inhibiting 50% of the activity of the enzyme, were determined using microsomes from the human placenta as source of the enzyme according to the tritiated water method described by PURBA et al (1990).

The most active compounds deliver an IC₅₀ close to 1 nanomolar.

### Example C

### Study of Cellular Cytotoxicity

The protocol of the study of cellular cytotoxicity is adapted after MOSMANN (1983).

It consists of the transformation of MTT to formazan by mitochondrial succinate dehydrogenase. This test is done on E293 cells of human embryonic kidney which do not express aromatase.

The results showed that the compounds are not cytotoxic.

### Example D

### Study of the Activity In Vivo

The activity in vivo of aromatase inhibition by compounds of formula (I) according to the invention has been tested according to the model established by Bharnagar et al. (1990).

In general, immature female rats of the Sprague-Dawley line of a weight ranging from 40 to 50 g have been treated with a dose of androstenedione at 30 mg/kg for 4 days, in the absence or in the presence of doses of various compounds of formula (I).

Four hours after administration of an aromatase inhibitor, the rats were sacrificed. Their uteruses were removed, cleared of adhering fat and conjunctive tissue, next the uteruses were weighed (wet weight). The dry weight of the uteruses was determined the following day after a step of drying for one night at 80° C.

The detailed results of the activity in vitro and in vivo of various aromatase inhibitors of formula (I) according to the invention are presented in table V, in the present description.

The results show that the compounds of formula (I) according to the invention induce a reduction of uterine hypertrophy induced by androstenedione which is dependent on the dose of compound of formula (I) used, with, for certain compounds of formula (I), an almost complete inhibition of the uterine hypertrophy induced by androstenedione.

TABLE I-A
6-ACYL-BENZAZINONES AND 7-ACYL-BENZOTHIAZINONES
6-Acyl-benzoxazolinones, 6-acyl-benzothiazolinones, 6-acyl-benzoxazinones,
6-acyl-benzothiazinones and 7-acyl-benzothiazinones

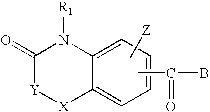

| Ex- | | | | | | | | |
|---|---|---|---|---|---|---|---|---|
| ample | R₁ | X | Y | Z | B | Molecule | F ° C. | Method |
| 1a | H | O | — | H | 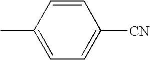 | 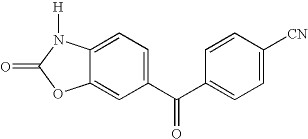 | 260-261 | B (AlCl₃/DMF) |
| 2a | CH₃ | O | — | H | 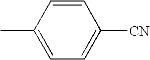 | 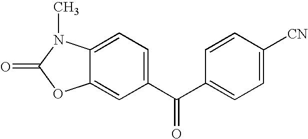 | 202-204 | B |
| 3a | H | O | — | H | 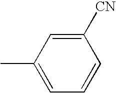 | 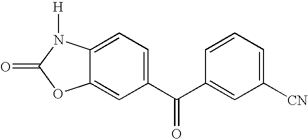 | 260-261 | B |
| 4a | CH₃ | O | — | H | 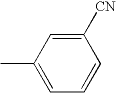 | 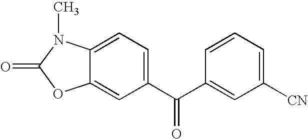 | 200-201 | B |
| 5a | CH₃ | O | — | H | 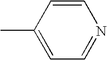 | 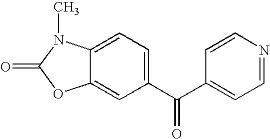 | 181-182 | B |
| 6a | CH₃ | O | — | H | 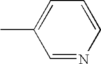 | 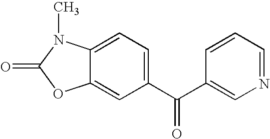 | 163-164 | B |
| 7a | H | S | — | H | 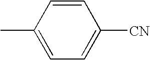 | 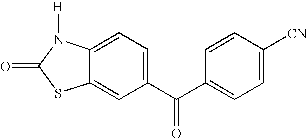 | 205-209 | B |
| 8a | CH₃ | S | — | H | 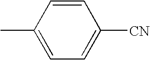 | 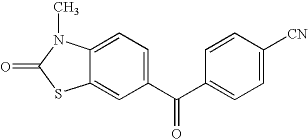 | 196-199 | B |
| 9a | CH₂CH₃ | S | — | H | 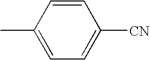 | 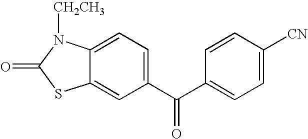 | 136-138 | B |
| 10a | H | CH₂ | — | H | 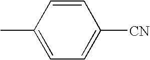 | 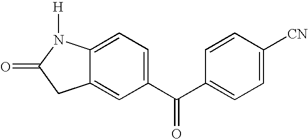 | 250-253 | B |
| 11a | H | O | CH₂ | H | 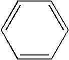 | 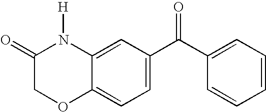 | 182-185 | A. (PPA) |
| 12a | CH₃ | O | CH₂ | H | 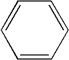 | 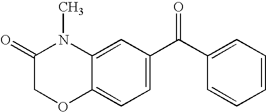 | 173-176 | A. |
| 13a | H | O | CH₂ | H | 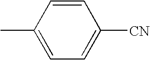 | 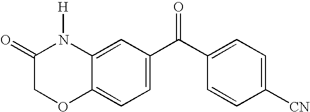 | 280-283 | B |
| 14a | CH₃ | O | CH₂ | H | 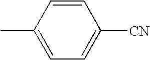 | 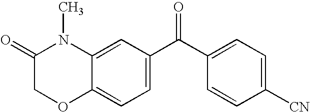 | 208-211 | B |
| 15a | H | S | CH₂ | H | 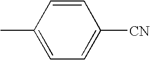 | 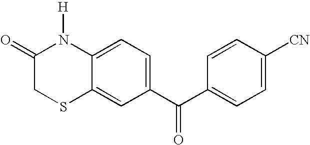 | 261-263 | B |
| 16a | CH₃ | S | CH₂ | H | 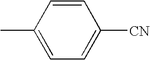 | 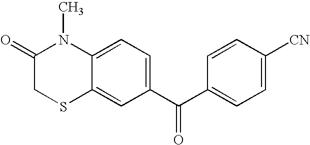 | 179-180 | B |
| 17a | H | O | — | H | 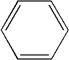 | 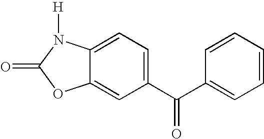 | 169-170 | A (PPA) |
| 18a | CH₃ | O | — | H | 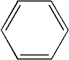 | 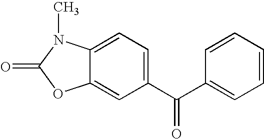 | 147-148 | A. |
| 19a | H | S | — | H | 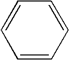 | 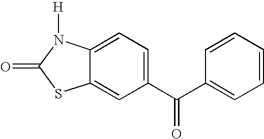 | 216-217 | A |
| 20a | CH₃ | S | — | H | 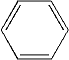 | 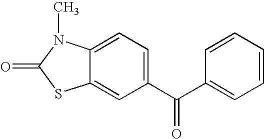 | 148-149 | A |
| 21a | CH₃ | O | — | H | 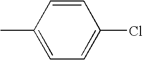 | 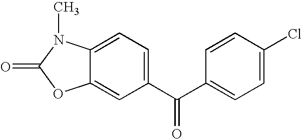 | 190-191 | A |
| 22a | CH₃ | S | — | H | 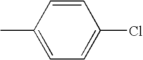 | 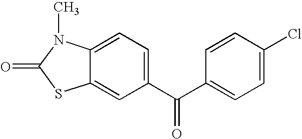 | 176-177 | A. |
| 23a | H | S | — | H | 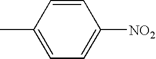 | 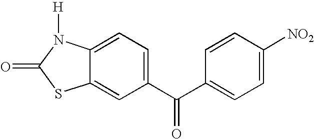 | 260-265 | B |
| 24a | H | O | CH₂ | H | 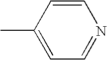 | 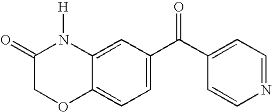 | 281-282 | B |
| 25a | H | S | CH₂ | H | 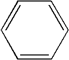 | 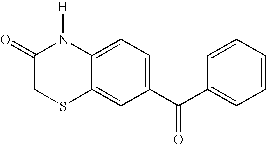 | 194-196 | B |

TABLE I-B
6-ACYL-BENZAZINONES
6-acyl-benzothiazolinones, 6-acyl-benzoselenazolinones

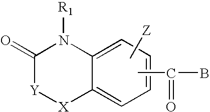

| Ex- | | | | | | | | | |
|---|---|---|---|---|---|---|---|---|---|
| am- | | | | | | Iso- | | | |
| ple | R₁ | X | Y | Z | B | mer | Molecule | F ° C. | Method |
| 1 | H | S | — | H | 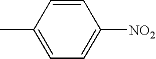 | 6 | 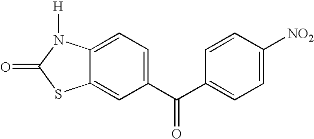 | 260-265 | B (AlCl₃/DMF) |
| 2 | CH₂CH₃ | S | — | H | 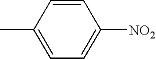 | 6 | 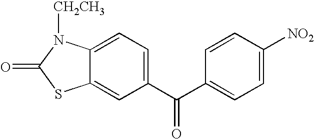 | 148-152 | N-alkyl |
| 3 | H | Se | — | H | 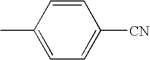 | 6 | 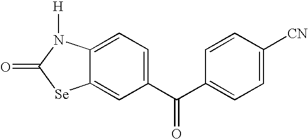 | 230-232 | B |
| 4 | CH₃ | Se | — | H | 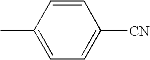 | 6 | 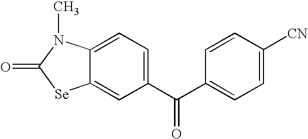 | 205-210 | B |
| 5 | CH₂CH₃ | Se | — | H | 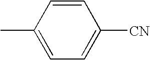 | 6 | 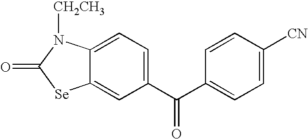 | 130-135 | N-alkyl |
| 6 | H | Se | — | H | 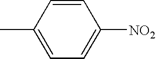 | 6 | 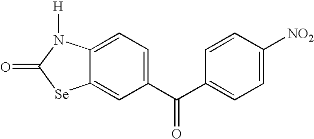 | 241-245 | B |
| 7 | CH₃ | Se | — | H | 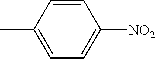 | 6 | 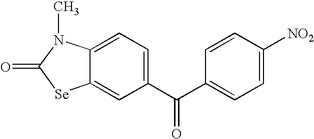 | 151-155 | N-alkyl |
| 8 | CH₂CH₃ | Se | — | H | 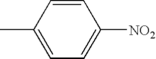 | 6 | 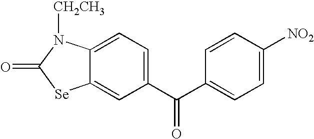 | 97-102 | N-alkyl |

TABLE II
5 and 7-ACYL-BENZAZINONES
5-Acyl-benzoxazolinones, 7-acyl-benzoxazinones

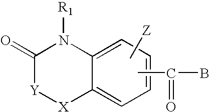

| Ex- | | | | | | | | Pre- |
|---|---|---|---|---|---|---|---|---|
| ample | R₁ | X | Y | Z | B | Molecule | F ° C. | paration |
| 26a | H | O | — | H | 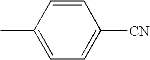 | 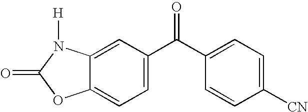 | 250-253 | 2 |
| 27a | H | O | — | H | 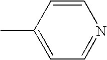 | 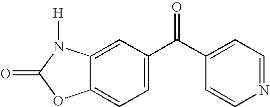 | 307-310 | 2 |
| 28a | H | O | — | 6-OCH₃ | 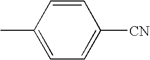 | 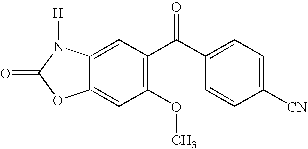 | 224-226 | 2 |
| 29a | H | O | — | H | 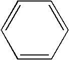 | 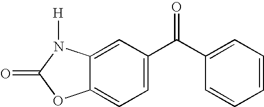 | 153-160 | 2 |
| 30a | CH₃ | O | — | H | 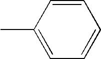 | 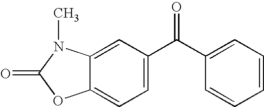 | 152-156 | 2 |
| 31a | CH₃ | O | — | H | 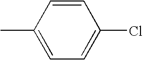 | 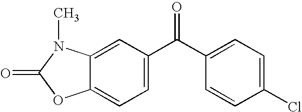 | 163-164 | 2 |
| 32a | H | O | CH₂ | H | 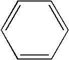 | 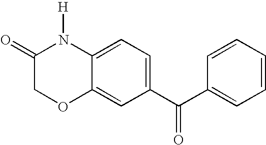 | 210-213 | 3 |
| 33a | CH₃ | O | CH₂ | H | 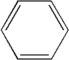 | 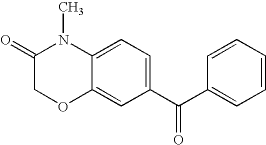 | 117-119 | 3 |

TABLE III-A
REDUCED DERIVATIVES
Hydroxyarylmethyl benzazinones

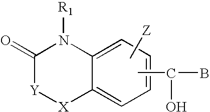

| Ex- | | | | | | | |
|---|---|---|---|---|---|---|---|
| ample | R₁ | X | Y | Z | B | Molecule | F ° C. |
| 1b | H | O | — | H | 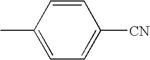 | 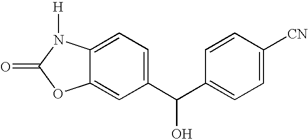 | 195-197 |
| 2b | CH₃ | O | — | H | 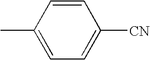 | 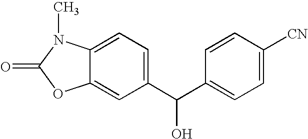 | 145-146 |
| 3b | H | O | — | H | 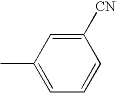 | 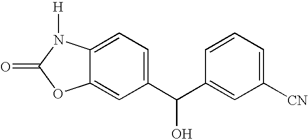 | 130-131 |
| 4b | CH₃ | O | — | H | 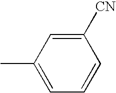 | 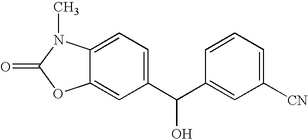 | 83-85 |
| 5b | CH₃ | O | — | H | 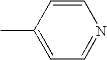 | 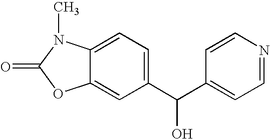 | 243-245 |
| 6b | CH₃ | O | — | H | 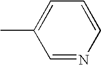 | 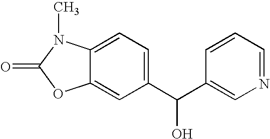 | 157-158 |
| 7b | H | S | — | H | 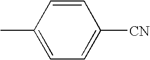 | 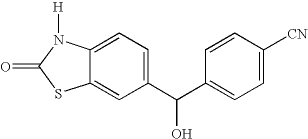 | 202-203 |
| 8b | CH₃ | S | — | H | 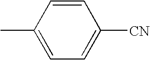 | 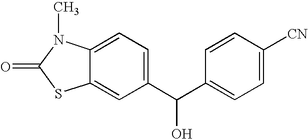 | 196-197 |
| 9b | CH₂CH₃ | S | — | H | 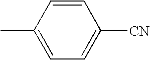 | 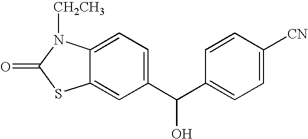 | 146-150 |
| 10b | H | CH₂ | — | H | 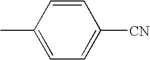 | 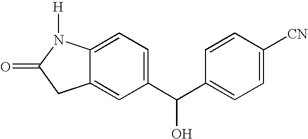 | 178-180 |
| 11b | H | O | CH₂ | H | 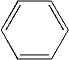 | 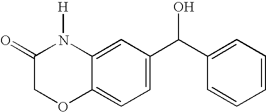 | 180-182 |
| 12b | CH₃ | O | CH₂ | H | 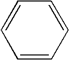 | 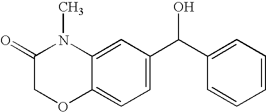 | unstable |
| 13b | H | O | CH₂ | H | 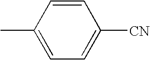 | 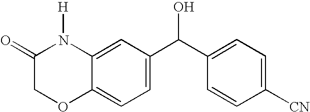 | 156-160 |
| 14b | CH₃ | O | CH₂ | H | 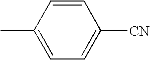 | 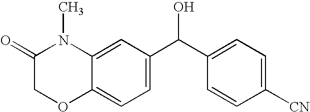 | 115-118 |
| 15b | H | S | CH₂ | H | 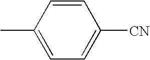 | 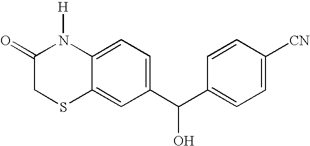 | 238-240 |
| 16b | CH₃ | S | CH₂ | H | 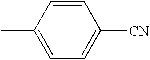 | 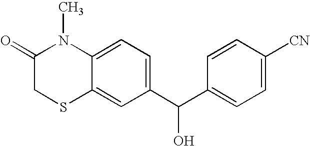 | 115-118 |
| 17b | H | O | — | H | 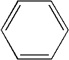 | 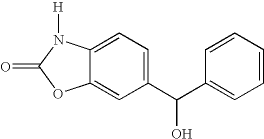 | 143-144 |
| 18b | CH₃ | O | — | H | 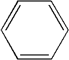 | 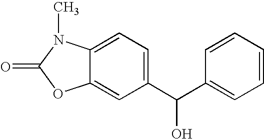 | 119-120 |
| 19b | H | S | — | H | 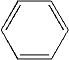 | 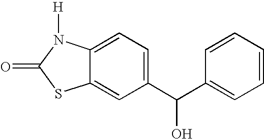 | 159-160 |
| 20b | CH₃ | S | — | H | 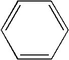 | 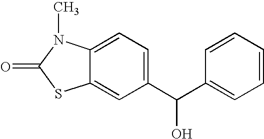 | 127-129 |
| 21b | CH₃ | O | — | H | 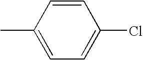 | 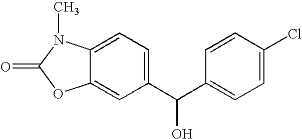 | 154-155 |
| 22b | CH₃ | S | — | H | 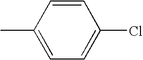 | 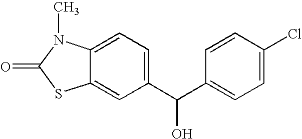 | 152-155 |
| 23b | H | S | — | H | 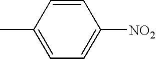 | 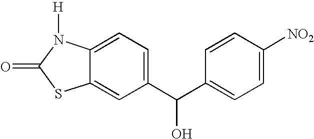 | 208-212 |
| 24b | H | O | CH₂ | H | 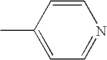 | 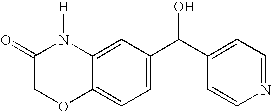 | 257-260 |
| 25b | H | S | CH₂ | H | 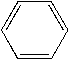 | 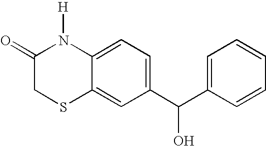 | 173-179 |
| 26b | H | O | — | H | 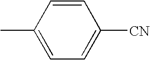 | 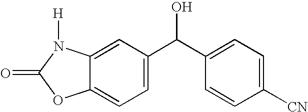 | 208-212 |
| 27b | H | O | — | H | 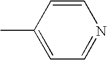 | 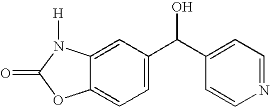 | 216-220 |
| 28b | H | O | — | 6-OCH₃ | 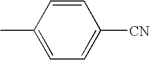 | 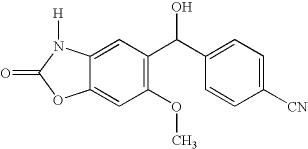 | 156-157 |
| 29b | H | O | — | H | 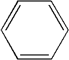 | 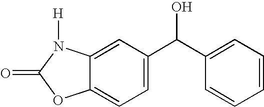 | 153-154 |
| 30b | CH₃ | O | — | H | 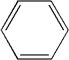 | 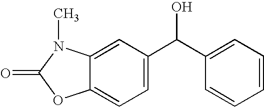 | 127-128 |
| 31b | CH₃ | O | — | H | 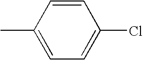 | 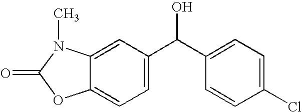 | 149-153 |
| 32b | H | O | CH₂ | H | 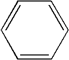 | 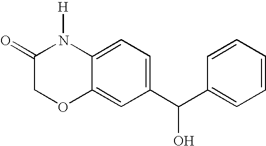 | 132-137 |
| 33b | CH₃ | O | CH₂ | H | 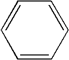 | 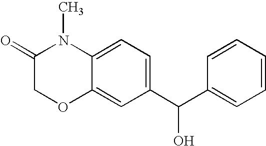 | 117-119 |

TABLE III-B
REDUCED DERIVATIVES
Hydroxylmethyl benzazinone

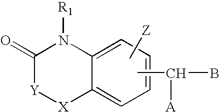

| Ex- | | | | | | | | |
|---|---|---|---|---|---|---|---|---|
| ample | R₁ | X | Y | Z | B | Isomer | Molecule | F ° C. |
| 1a | CH₂CH₃ | S | — | H | 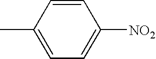 | 6 | 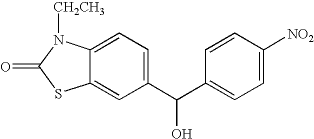 | 160-162 |
| 2a | H | Se | — | H | 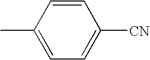 | 6 | 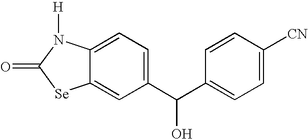 | 209-213 |
| 3a | CH₃ | Se | — | H | 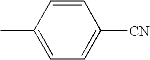 | 6 | 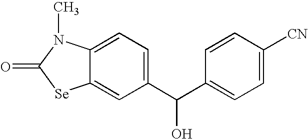 | 205-208 |
| 4a | CH₂CH₃ | Se | — | H | 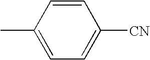 | 6 | 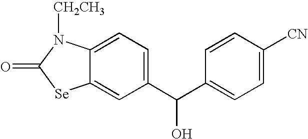 | 132-134 |
| 5a | CH₃ | Se | — | H | 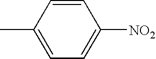 | 6 | 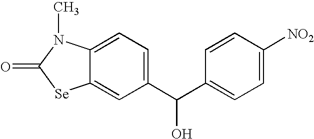 | 182-183 |
| 6a | CH₂CH₃ | Se | — | H | 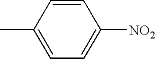 | 6 | 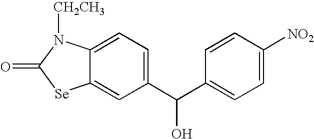 | 135-137 |
| 7a | CH₃ | S | — | H | 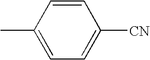 | 5 | 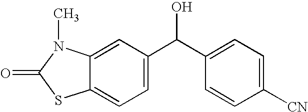 | 183-186 |
| 8a | CH₂CH₃ | S | — | H | 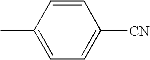 | 5 | 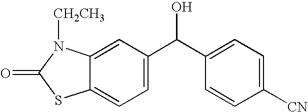 | 156-158 |

TABLE IV

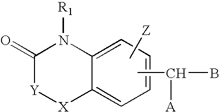

| Ex- | | | | | | | | | |
|---|---|---|---|---|---|---|---|---|---|
| ample | Code | R₁ | X | Y | Z | A | B | Molecule | F ° C. |
| 1 | PCH113 | H | O | — | H | 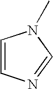 | 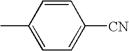 | 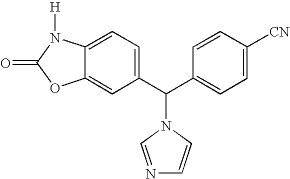 | 122-126 |
| 2 | PCH27 | CH₃ | O | — | H | 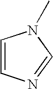 | 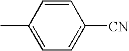 | 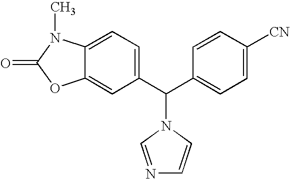 | 85-87 |
| 3 | PCH119 | H | O | — | H | 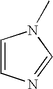 | 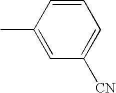 | 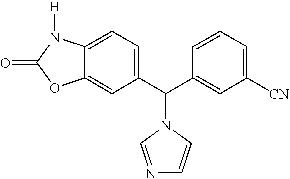 | 113-117 |
| 4 | PCH122 | CH₃ | O | — | H | 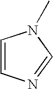 | 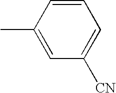 | 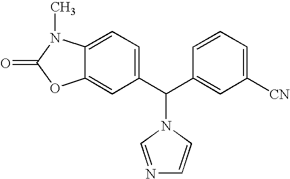 | 185-187 |
| 5 | PCH30 | CH₃ | O | — | H | 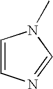 | 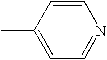 | 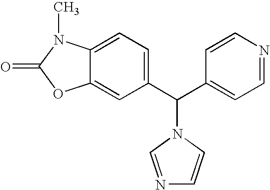 | 66-68 |
| 6 | PCH116 | CH₃ | O | — | H | 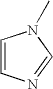 | 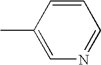 | 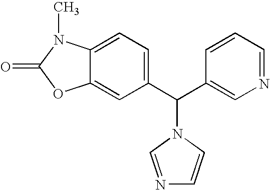 | 60-65 |
| 7 | PCH215 | H | S | — | H | 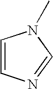 | 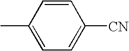 | 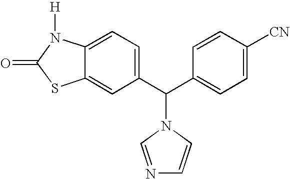 | 214-216 |
| 8 | PCH165 | CH₃ | S | — | H | 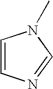 | 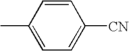 | 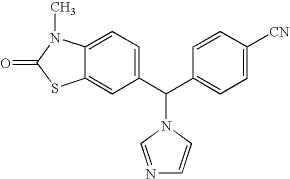 | 105-108 |
| 9 | PCH241 | CH₂CH₃ | S | — | H | 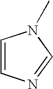 | 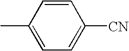 | 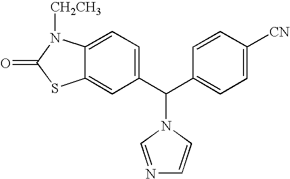 | 95-98 |
| 10 | PCH234 | H | CH₂ | — | H | 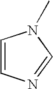 | 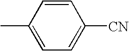 | 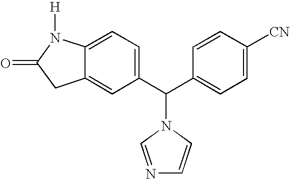 | 200-209 |
| 11 | PCH218 | H | O | CH₂ | H | 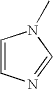 | 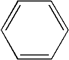 | 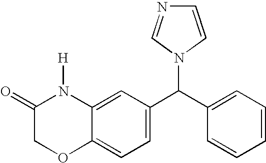 | 139-143 |
| 12 | PCH213 | CH₃ | O | CH₂ | H | 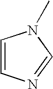 | 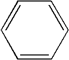 | 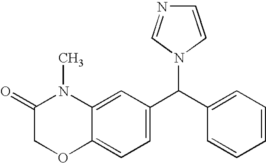 | 123-125 |
| 13 | PCH225 | H | O | CH₂ | H | 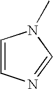 | 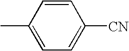 | 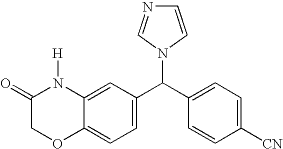 | 135-140 |
| 14 | PCH222 | CH₃ | O | CH₂ | H | 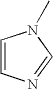 | 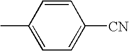 | 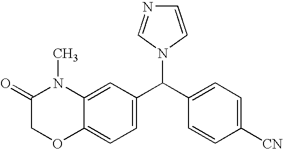 | 80-87 |
| 15 | PCH229 | H | S | CH₂ | H | 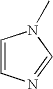 | 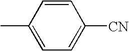 | 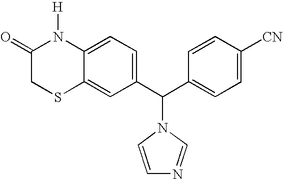 | 150-155 |
| 16 | PCH240 | CH₃ | S | CH₂ | H | 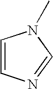 | 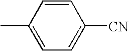 | 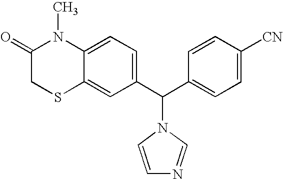 | 74-80 |
| 17 | PCH128 | H | O | — | H | 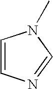 | 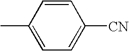 | 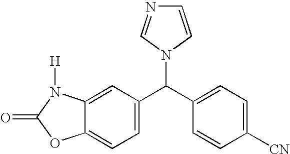 | 128-132 |
| 18 | PCH129 | H | O | — | H | 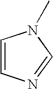 | 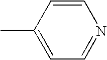 | 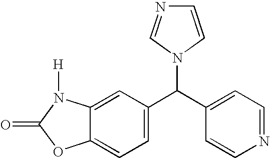 | 75-80 |
| 19 | GCA36 | H | O | — | 6-OCH₃ | 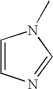 | 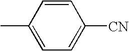 | 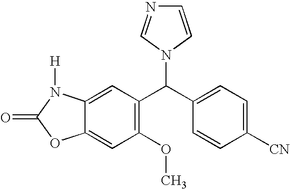 | 165-160 |
| 20 | PCH216 | H | S | — | H | 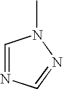 | 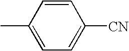 | 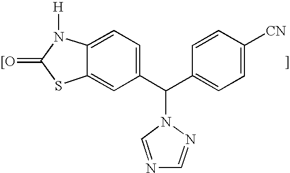 | 127-130 |
| 21 | PCH158 | CH₃ | S | — | H | 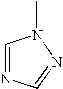 | | 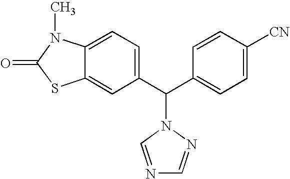 | 165-168 |
| 22 | PCH230 | H | S | CH₂ | H | 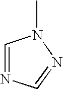 | 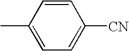 | 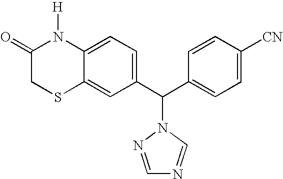 | 215-218 |
| 23 | PCH231 | CH₃ | S | CH₂ | H | 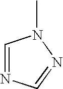 | 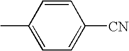 | 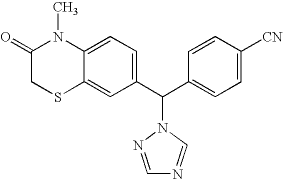 | 95-100 |
| 24 | PCH211 | H | O | CH₂ | H | 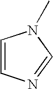 | 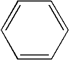 | 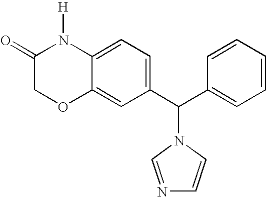 | 203-206 |
| 25 | PCH10 | H | O | — | H | 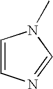 | 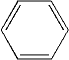 | 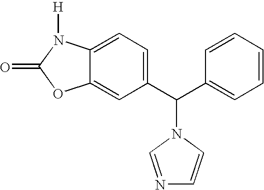 | 193-195 |
| 26 | AL22 | CH₃ | O | — | H | 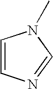 | 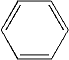 | 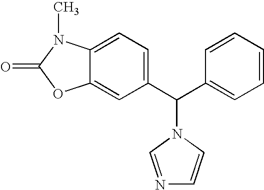 | 73-74 |
| 27 | PCH15 | CH₃ | O | — | H | 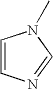 | 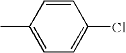 | 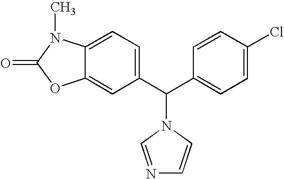 | 76-78 |
| 28 | PCH21 | CH₃ | O | — | H | 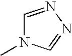 | 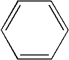 | 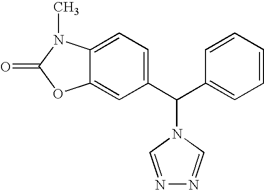 | 225-226 |
| 29 | PCH20 | CH₃ | O | — | H | 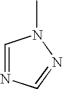 | 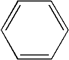 | 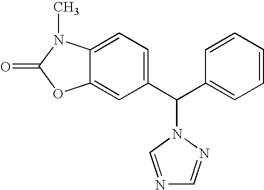 | 77-79 |
| 30 | PCH124 | H | O | — | H | 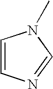 | 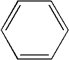 | 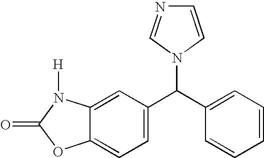 | 108-111 |
| 31 | PCH31 | CH₃ | O | — | H | 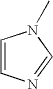 | 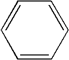 | 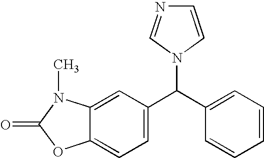 | 133-135 |
| 32 | PCH183 | CH₃ | O | — | H | 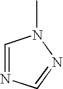 | 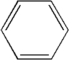 | 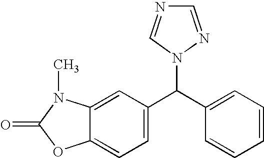 | 135-138 |
| 33 | PCH160 | CH₃ | O | — | H | 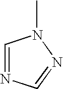 | 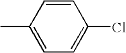 | 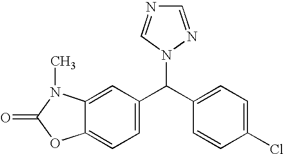 | 70-74 |
| 34 | GCA37 | H | O | — | 6-OCH₃ | 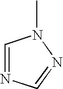 | 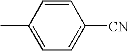 | 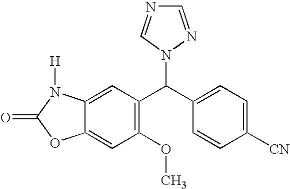 | 125-130 |
| 35 | PCH100 | H | S | — | H | 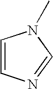 | 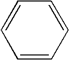 | 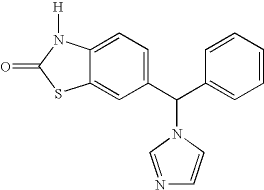 | 55-60 |
| 36 | PCH28 | CH₃ | S | — | H | 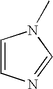 | 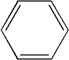 | 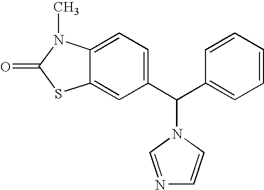 | 65-68 |
| 37 | PCH208 | CH₃ | S | — | H | 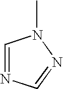 | 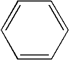 | 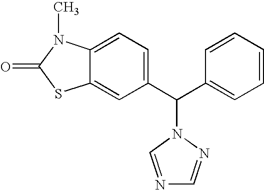 | 150-154 |
| 38 | PCH164 | CH₃ | S | — | H | 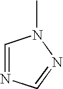 | 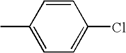 | 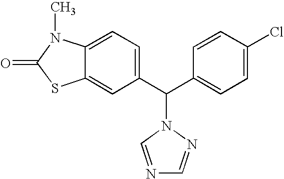 | 106-112 |
| 39 | PCH249 | H | S | — | H | 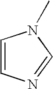 | 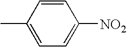 | 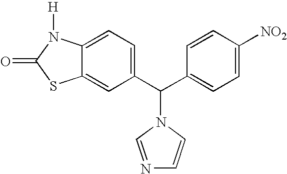 | 238-241 |
| 40 | PCH19 | CH₃ | O | CH₂ | H | 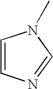 | 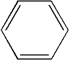 | 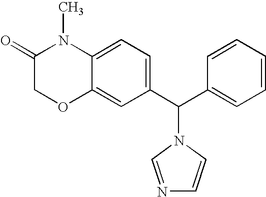 | 66-68 |
| 41 | PCH210 | CH₃ | O | CH₂ | H | 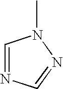 | 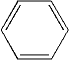 | 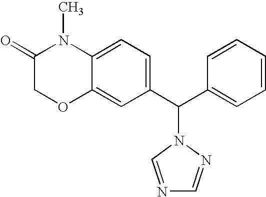 | 160-164 |
| 42 | PCH214 | CH₃ | O | CH₂ | H | 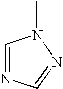 | 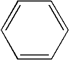 | 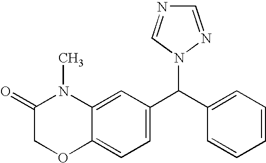 | 140-150 |
| 43 | PCH227 | H | S | CH₂ | H | 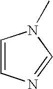 | 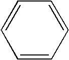 | 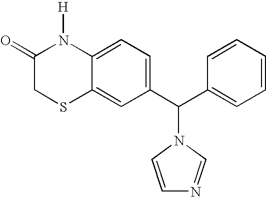 | 187-189 |

| Ex- | | | | | | | | Iso- | | |
|---|---|---|---|---|---|---|---|---|---|---|
| ample | Code | R₁ | X | Y | Z | A | B | mer | Molecule | F ° C. |
| 44 | PCH243 | CH₂CH₃ | S | — | H | 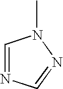 | 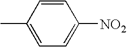 | 6 | 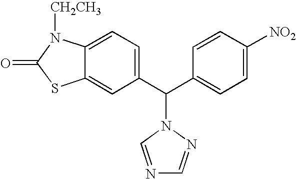 | 79-83 |
| 45 | PCH302 | H | Se | — | H | 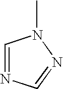 | 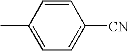 | 6 | 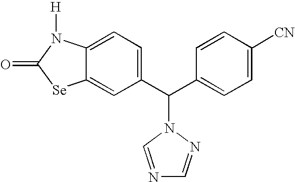 | 223-226 |
| 46 | PCH300 | CH₃ | Se | — | H | 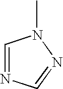 | 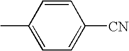 | 6 | 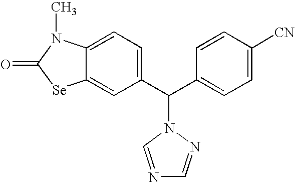 | 154-158 |
| 47 | PCH303 | CH₂CH₃ | Se | — | H | 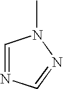 | 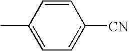 | 6 | 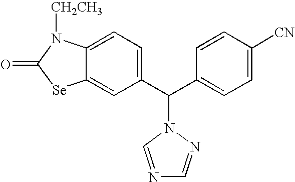 | 95-98 |
| 48 | PCH304 | CH₃ | Se | — | H | 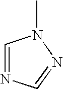 | 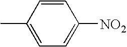 | 6 | 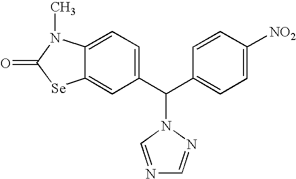 | 190-195 |
| 49 | PCH305 | CH₂CH₃ | Se | — | H | 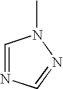 | 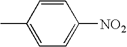 | 6 | 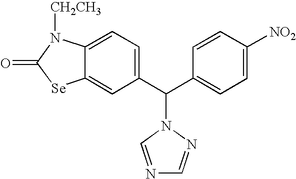 | 79-82 |
| 50 | PCH163 | CH₃ | S | — | H | 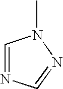 | 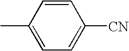 | 5 | 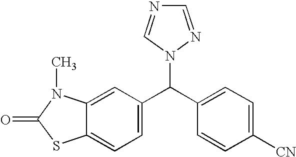 | 122-125 |
| 51 | PCH246 | CH₂CH₃ | S | — | H | 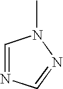 | 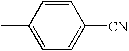 | 5 | 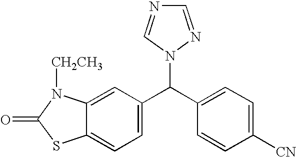 | 125-127 |

TABLE V
Results of tests in vitro and in vivo of compounds of formula (I) according to the invention

| | | Activity | |
|---|---|---|---|
| | | In vitro | |
| Code | Compound | IC₅₀(nM) | % inhibition to doses (μg/Kg) |
| Letrozole | 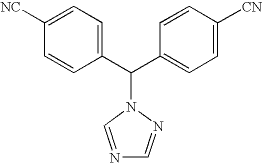 | 4.23 | 66%(1) 57, 59%(1) 74%(3) 91, 86%(5) 90%(10) 94, 89%(10) |
| (s)-Fadrozole | 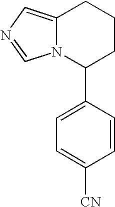 | 61(h) 260(e) | |

BENZOXAZOLINONIC DERIVATIVES
Benzoxazolinonic derivatives substituted
in position 6

| | | | |
|---|---|---|---|
| PCH10 | 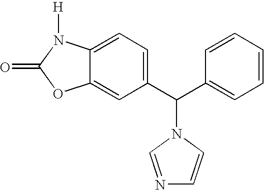 | 84.63(h) 103.3(e) | |
| AL22 | 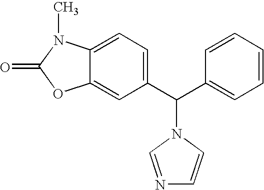 | 320(h) 340(e) | |
| PCH15 | 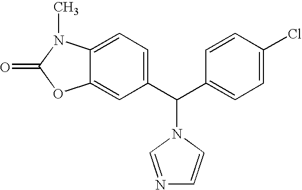 | >2000(h) nd(e) | |
| PCH30 | 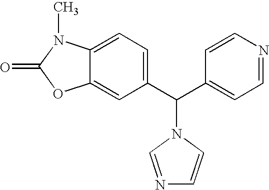 | 38.0(h) 477(e) | |
| PCH116 | 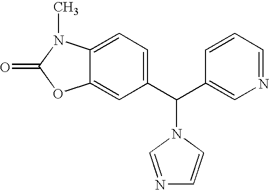 | 33.7(h) 34.6(e) | |
| PCH113 | 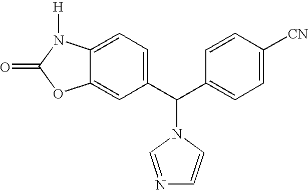 | 13.25(h) 14.6(e) | 19%(10) 50%(100) 94%(1000) |
| PCH27 | 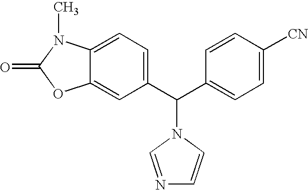 | 4.62(h) 72(e) | |
| PCH119 | 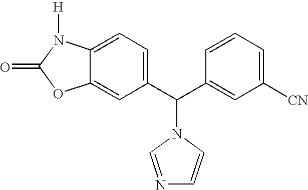 | 25.05(h) 27.7(e) | |
| PC122 | 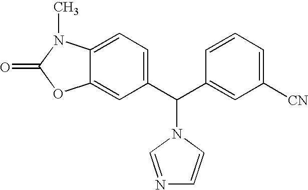 | 18.63(h) 23.25(e) | 39%(10) 58%(100) 92%(1000) |
| PCH21 | 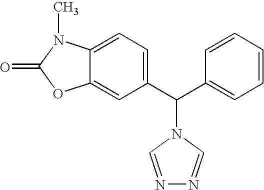 | >3000(h) nd(e) | |
| PCH20 | 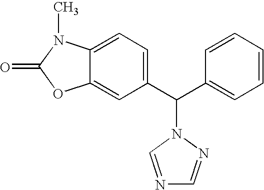 | >3000(h) nd(e) | |

Benzoxazolinonic derivatives substituted
in position 5

| | | | |
|---|---|---|---|
| PCH124 | 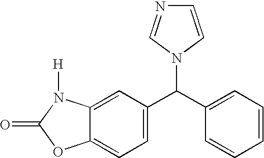 | 14.95(h) 14.1(e) | 34%(10) 71%(100) 92%(1000) |
| PCH31 | 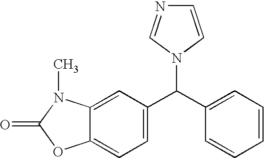 | 46.6(h) 50.1(e) | |
| PC11129 | 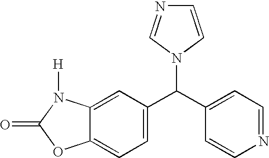 | 26.8 | |
| PC11128 | 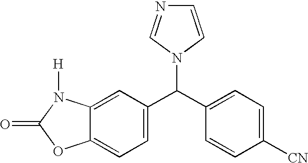 | 5.83 | 29%(1) 29%(10) 53%(100) |
| GCA36 | 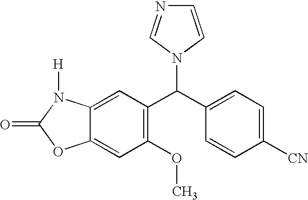 | 19.9 | |
| PCH183 | 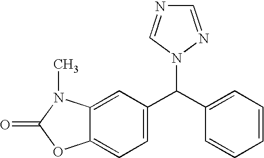 | 1813 | |
| PCH160 | 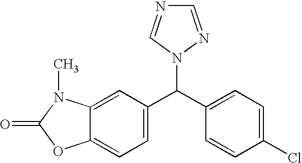 | 18.7 | |
| PCH195 | 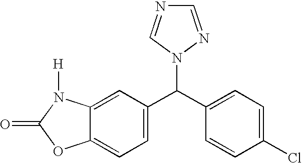 | 17.1 | |
| PCH196 | 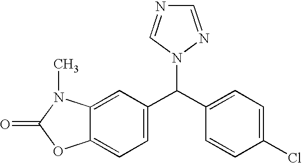 | 24.9 | |
| GCA37 | 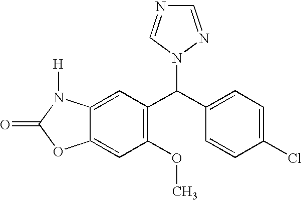 | 328 | |

Benzothiazolinonic derivatives
Benzothiazolinonic derivatives substituted
in position 6

| | | | |
|---|---|---|---|
| PCH100 | 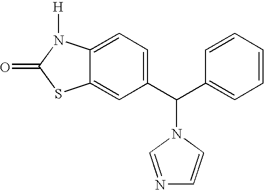 | 33.65(h) 34.0(e) | |
| (+/−)PCH28 (E1)PCH28 (E2) PCH28 | 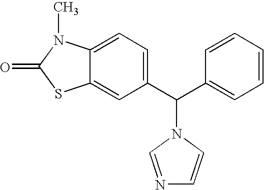 | 12.1(h) 23.4(e) 24.35(h) 24.9(e) 26.43(h) 22.6(e) | 13%(10) 25%(100) 75%(1000) |
| PC11215 | 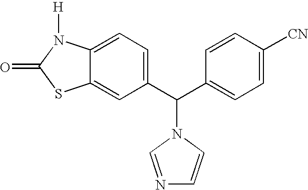 | 4.04 | 0%(1)56%(10) 90%(100) |
| PCH165 (+/−) CD4 PCH165 (+)CD4 PCH165 (−)CD4 | 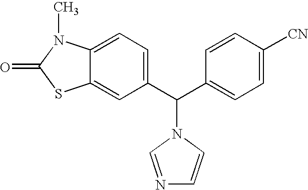 | 4.54 8.81 4.94 | 22%(1) 23%(10) 66%(100) |
| PCH241 | 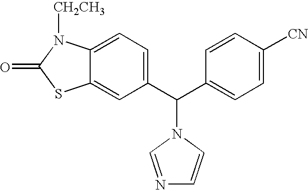 | 4.29 | 18%(1) 37%(3) 16%(10) |
| PCH216 | 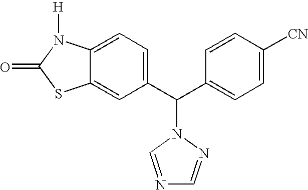 | 7.51 | 21%(1) 32%(10) 76%(100) |
| PCH158 (PCH190) | 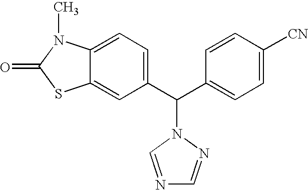 | 8.71 | 54, 60%(1) 56, 74%(10) 68, 100%(100) |
| PCH260 | 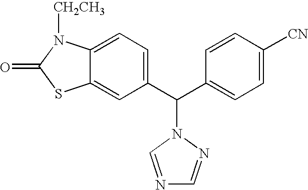 | 4.49 | 32%(1) 50%(10) 90%(100) |
| PCH258 | 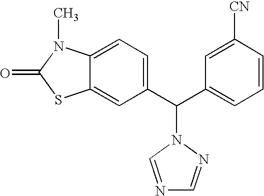 | 31.7 | |
| PCH259 | 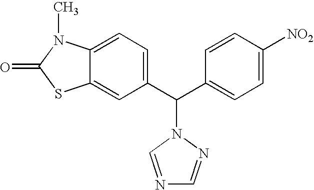 | 3.05 | 31%(1) 63%(10) 88%(100) |
| PCH243 | 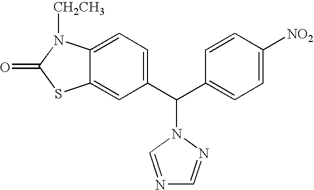 | 3.99 | |
| PCH248 | 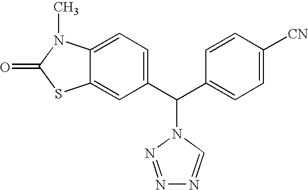 | 11.8 | |

Benzothiazolinonic derivatives
substituted in position 5

| | | | |
|---|---|---|---|
| PCH132 | 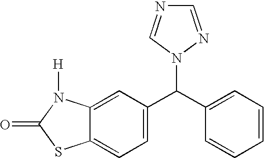 | 178 | |
| PCH134 | 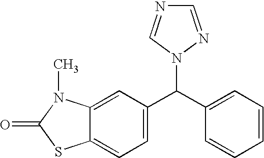 | 179 | |
| PCH163 | 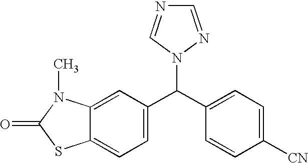 | 5.78 | 57%(1) 83%(10) 95%(100) |
| PCH246 | 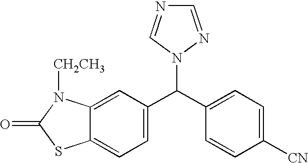 | 5.51 | 22%(1) 45%(10) 91%(100) |

Benzothiazolinonic derivatives
Selenazolinonic derivatives substituted
in position 6

| | | | |
|---|---|---|---|
| PCH300 | 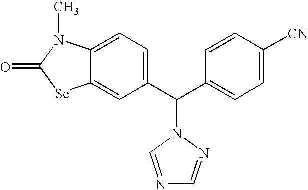 | 4.64 | 49%(1) 86%(10) 91%(100) |
| PCH302 | 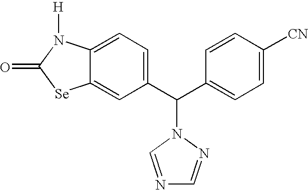 | 6.53 | 45%(1) 20%(10) 63%(100) |
| PC11303 | 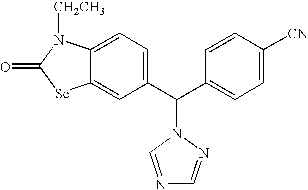 | 3.99 | 38%(1) 60%(10) 71%(100) |
| PCH304 | 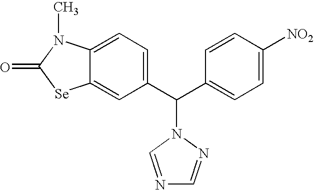 | 3.64 | |
| PCH305 | 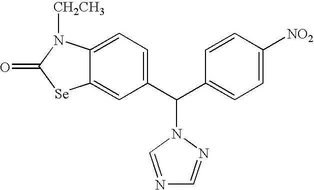 | 3.70 | |

Benzothiazolinonic derivatives
Benzothiazolinonic derivatives substituted
in position 7

| | | | |
|---|---|---|---|
| PCH19 | 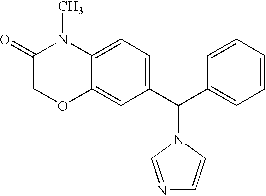 | 52.48(h) 59.87(e) | |
| PCH211 | 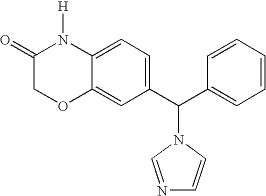 | 74.4 | |
| PCH218 | 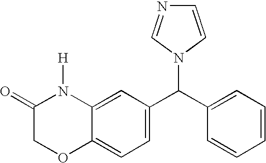 | 65.5 | |
| PCH213 | 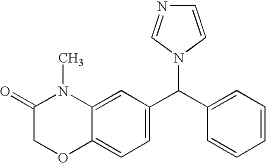 | 5.64 | 0%(1) 3%(10) 5%(100) |
| PCH225 | 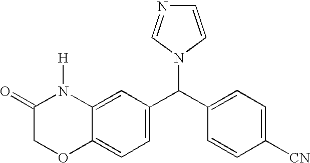 | 9.90 | |
| PCH222 | 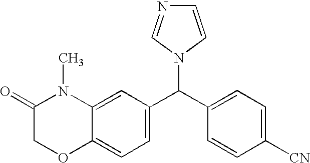 | 3.44 | 0%(1) 22%(3) 32%(10) |
| PCH223 | 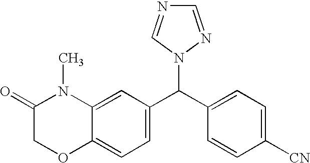 | | 4%(1) 22%(10) 66%(100) |

Benzothiazolinonic derivatives
Benzothiazolinonic derivatives substituted
in position 7

| | | | |
|---|---|---|---|
| PCH227 | 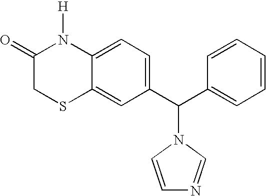 | 55.1 | |
| PCH229 | 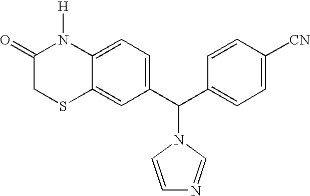 | 13.8 | 11%(10) 42%(100) 83%(1000) |
| PCH240 | 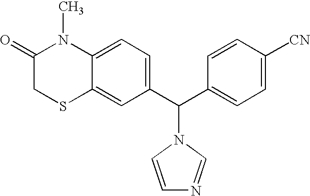 | 5.38 | |
| PCH230 | 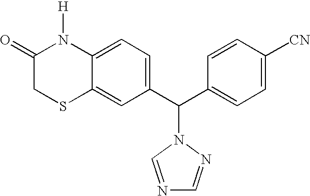 | 34.8 | 22%(100) 2%(10) 74%(1000) |
| PCH231 | 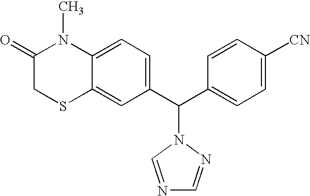 | 56.6 | |

1.-6. (canceled)

7. A method comprising obtaining a compound of formula (I) or enantiomer, diastereomer, or salt thereof: 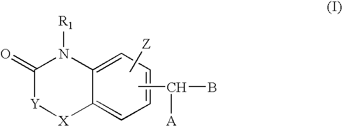
wherein:
R₁ is an atom of hydrogen or a linear or branched alkyl (C₁-C₆), alkenyl (C₁-C₆), or alkynyl (C₁-C₆) radical,
X is:
an atom of oxygen, of sulphur or of selenium and Y is a single bond; or
an atom of sulphur and Y is a CH₂ group;
Z is an atom of hydrogen or of halogen, or a linear or branched hydroxy or alkoxy group;
A is an imidazole, triazole or tetrazole nucleus; and
B is a phenyl, naphthyl, biphenyl or a monocyclic or bicyclic heteroaryl group having 5 to 10 bonds and comprising 1 to 3 heteroatoms, wherein the phenyl, naphthyl, biphenyl, or heteroaryl group is non-substituted or substituted by 1 to 3 alkyl (C₁-C₆), alkoxy (C₁-C₆), carboxy, formyl, amino, amido, ester, nitro, cyano, or trifluoromethyl, groups and/or halogens; and
administering the compound, enantiomer, diastereomer, or salt to a subject with cancer or psoriasis.

8. The method of claim 7, wherein X is an atom of sulphur and Y is a CH₂ group substituted by one or two lower alkyl groups.

9. The method of claim 7, wherein B is:
an unsubstituted benzene or benzene substituted in the meta or para position by a cyano or nitro group or an atom of chlorine; or
a pyridine heterocycle.

10. The method of claim 7, wherein R₁ is an atom of hydrogen or a methyl group.

11. The method of claim 7, wherein Z is an atom of hydrogen or a methoxy group.

12. The method of claim 7, wherein A is a 1,3-imidazolyl or 1,2,4 triazolyl group.

13. The method of claim 7, wherein the compound of formula (I) is:
5-[(4-Cyanophenyl)(1H-imidazol-1-yl)methyl]-1,3-benzoxazol-2(3H)-one;
6-[(4-Cyanophenyl)(1H-imidazol-1-yl)methyl]-1,3-benzothiazol-2(3H)-one;
6-[(4-Cyanophenyl)(1H-imidazol-1-yl)methyl]-3-methyl-1,3-benzothiazol-2(3H)-one;
6-[(4-Cyanophenyl)(1H-1,2,4-triazol-1-yl)methyl]1-1,3-benzothiazol-2(3H)-one;
6-[(4-Cyanophenyl)(1H-1,2,4-triazol-1-yl)methyl]-3-methyl-1,3-benzothiazol-2(3H)-one;
6-[(4-Cyanophenyl)(1H-1,2,4-triazol-1-yl)methyl]-3-ethyl-1,3-benzothiazol-2(3H)-one;
6-[(4-Cyanophenyl)(1H-imidazol-1-yl)methyl]-1,4-benzoxazin-3(4H)-one;
6-[(4-Cyanophenyl)(1H-imidazol-1-yl)methyl]-4-methyl-1,4-benzoxazin-3(4H)-one;
7-[(4-Cyanophenyl)(1H-imidazol-1-yl)methyl]-4-methyl-1,4-benzothiazin-3(4H)-one;
3-Ethyl-6-[(4-nitrophenyl)(1H-1,2,4-triazol-1-yl)methyl]-1,3-benzothiazol-2(3H)-one;
4-[(2-oxo-2,3-dihydro-1,3-benzoselenazol-6-yl)(1H-1,2,4-triazol-1-yl)methyl]benzonitrile;
4-[(3-Methyl-2-oxo-2,3-dihydro-1,3-benzoselenazol-6-yl)(1H-1,2,4-triazol-1-yl)methyl]benzonitrile;
4-[(3-Ethyl-2-oxo-2,3-dihydro-1,3-benzoselenazol-6-yl)(1H-1,2,4-triazol-1-yl)methyl]benzonitrile;
3-Methyl-6-[(4-nitrophenyl)(1H-1,2,4-triazol-1-yl)methyl]-1,3-benzoselenazol-2(3H)-one;
3-Ethyl-6-[(4-nitrophenyl)(1H-1,2,4-triazol-1-yl)methyl]-1,3-benzoselenazol-2(3H)-one;
4-[(3-Methyl-2-oxo-2,3-dihydro-1,3-benzothiazol-5-yl)(1H-1,2,4-triazol-1-yl)methyl]benzonitrile; or
4-[(3-Ethyl-2-oxo-2,3-dihydro-1,3-benzothiazol-5-yl)(1H-1,2,4-triazol-1-yl)methyl]benzonitrile.

14. The method of claim 7, wherein the subject has cancer.

15. The method of claim 7, wherein the subject has psoriasis.

16. The method of claim 7, wherein the subject is a human.

17. A pharmaceutical composition comprising a compound of formula (I) or enantiomer, diastereomer, or salt thereof: 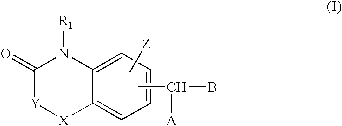
wherein:
R₁ is an atom of hydrogen or a linear or branched alkyl (C₁-C₆), alkenyl (C₁-C₆), or alkynyl (C₁-C₆) radical,
X is:
an atom of oxygen, of sulphur or of selenium and Y is a single bond; or
an atom of sulphur and Y is a CH₂ group;
Z is an atom of hydrogen or of halogen, or a linear or branched hydroxy or alkoxy group;
A is an imidazole, triazole or tetrazole nucleus; and
B is a phenyl, naphthyl, biphenyl or a monocyclic or bicyclic heteroaryl group having 5 to 10 bonds and comprising 1 to 3 heteroatoms, wherein the phenyl, naphthyl, biphenyl, or heteroaryl group is non-substituted or substituted by 1 to 3 alkyl (C₁-C₆), alkoxy (C₁-C₆), carboxy, formyl, amino, amido, ester, nitro, cyano, or trifluoromethyl, groups and/or halogens;
in a pharmaceutically acceptable formulation.

18. The composition of claim 17, wherein X is an atom of sulphur and Y is a CH₂ group substituted by one or two lower alkyl groups.

19. The composition of claim 17, wherein B is:
an unsubstituted benzene or benzene substituted in the meta or para position by a cyano or nitro group or an atom of chlorine; or
a pyridine heterocycle.

20. The composition of claim 17, wherein R₁ is an atom of hydrogen or a methyl group.

21. The composition of claim 17, wherein Z is an atom of hydrogen or a methoxy group.

22. The composition of claim 17, wherein A is a 1,3-imidazolyl or 1,2,4 triazolyl group.

23. The composition of claim 17, wherein the compound of formula (I) is:
5-[(4-Cyanophenyl)(1H-imidazol-1-yl)methyl]-1,3-benzoxazol-2(3H)-one;
6-[(4-Cyanophenyl)(1H-imidazol-1-yl)methyl]-1,3-benzothiazol-2(3H)-one;
6-[(4-Cyanophenyl)(1H-imidazol-1-yl)methyl]-3-methyl-1,3-benzothiazol-2(3H)-one;
6-[(4-Cyanophenyl)(1H-1,2,4-triazol-1-yl)methyl]-1,3-benzothiazol-2(3H)-one;
6-[(4-Cyanophenyl)(1H-1,2,4-triazol-1-yl)methyl]-3-methyl-1,3-benzothiazol-2(3H)-one;
6-[(4-Cyanophenyl)(1H-1,2,4-triazol-1-yl)methyl]-3-ethyl-1,3-benzothiazol-2(3H)-one;
6-[(4-Cyanophenyl)(1H-imidazol-1-yl)methyl]-1,4-benzoxazin-3(4H)-one;
6-[(4-Cyanophenyl)(1H-imidazol-1-yl)methyl]-4-methyl-1,4-benzoxazin-3(4H)-one;
7-[(4-Cyanophenyl)(1H-imidazol-1-yl)methyl]-4-methyl-1,4-benzothiazin-3(4H)-one;
3-Ethyl-6-[(4-nitrophenyl)(1H-1,2,4-triazol-1-yl)methyl]-1,3-benzothiazol-2(3H)-one;
4-[(2-oxo-2,3-dihydro-1,3-benzoselenazol-6-yl)(1H-1,2,4-triazol-1-yl)methyl]benzonitrile;
4-[(3-Methyl-2-oxo-2,3-dihydro-1,3-benzoselenazol-6-yl)(1H-1,2,4-triazol-1-yl)methyl]benzonitrile;
4-[(3-Ethyl-2-oxo-2,3-dihydro-1,3-benzoselenazol-6-yl)(1H-1,2,4-triazol-1-yl)methyl]benzonitrile;
3-Methyl-6-[(4-nitrophenyl)(1H-1,2,4-triazol-1-yl)methyl]-1,3-benzoselenazol-2(3H)-one;
3-Ethyl-6-[(4-nitrophenyl)(1H-1,2,4-triazol-1-yl)methyl]-1,3-benzoselenazol-2(3H)-one;
4-[(3-Methyl-2-oxo-2,3-dihydro-1,3-benzothiazol-5-yl)(1H-1,2,4-triazol-1-yl)methyl]benzonitrile; or
4-[(3-Ethyl-2-oxo-2,3-dihydro-1,3-benzothiazol-5-yl)(1H-1,2,4-triazol-1-yl)methyl]benzonitrile.
